(19) United States
(12) Patent Application Publication
(10) Pub. No.: US 2024/0150759 A1
(43) Pub. Date: May 9, 2024

(54) Splicing Modulators for the Treatment of Timothy Syndrome

(71) Applicant: The Board of Trustees of the Leland Stanford Junior University, Stanford, CA (US)

(72) Inventors: Sergiu P. Pasca, Stanford, CA (US); Xiaoyu Chen, Stanford, CA (US); Fikri Birey, Stanford, CA (US)

(21) Appl. No.: 18/496,554

(22) Filed: Oct. 27, 2023

Related U.S. Application Data
(60) Provisional application No. 63/422,567, filed on Nov. 4, 2022.

(51) Int. Cl. C12N 15/113; A61P 9/06

(52) U.S. Cl. CPC C12N 15/113; A61P 9/06; C12N 2310/11; C12N 2310/321

(57) ABSTRACT

The present disclosure provides methods of treating an individual having Timothy syndrome. Aspects of the methods include administering an effective dose of an agent to the individual, wherein the agent modulates the splicing of an 8 A or an 8 exon of CACNA1C. Also provided are compositions that find use in practicing embodiments of the methods.

### CROSS-REFERENCE TO RELATED APPLICATIONS

Pursuant to 35 U.S.C. § 119 (e), this application claims priority to the filing date of U.S. Provisional Patent Application Ser. No. 63/422,567 filed Nov. 4, 2022, the disclosure of which application is herein incorporated by reference.

### GOVERNMENT RIGHTS

This invention was made with Government support under contract RO1 MH115012 awarded by the National Institutes of Health. The Government has certain rights in the invention.

### REFERENCE TO AN ELECTRONIC SEQUENCE LISTING

The contents of the electronic sequence listing (STAN-2040_Seq_List.xml; Size: 33,258 bytes; and Date of Creation: Oct. 27, 2023) is herein incorporated by reference in its entirety.

### BACKGROUND OF THE INVENTION

Timothy syndrome type 1 (TS1) is a severe genetic disorder caused by the heterozygous c.1216G>A pathogenic variant in exon 8 A of CACNA1C, resulting in a p.G406R missense variant in the α1 subunit of the L-type voltage-gated calcium channel Caᵥ1.2. TS1 impacts multiple organ systems and is one of the most penetrant genetic etiologies of autism spectrum disorder (ASD) and epilepsy, and has high mortality. Variants in CACNA1C have also been strongly associated with other neuropsychiatric disorders, including schizophrenia, bipolar disorder, and attention deficit hyperactivity disorder, suggesting that Caᵥ1.2 is a key susceptibility factor for neuropsychiatric conditions.

Studies in hiPS cell-derived cardiomyocytes and neurons in 2D and 3D systems revealed that cells derived from individuals with TS1 have delayed voltage-gated channel inactivation and increased depolarization-induced calcium entry, which leads to abnormal excitability. Moreover, using human forebrain assembloids (hFA) generated by integrating human cortical organoids (hCO) and human subpallial organoids (hSO), it was previously described that defects in cortical interneuron migration. Specifically, it was discovered that TS1 interneurons undergo more frequent nucleokinetic saltations driven by enhanced GABA sensitivity, but the saltation length is reduced due to aberrant cytoskeleton function, leading to overall defective migration.

TS1-derived neurons have abnormally high levels of the CACNA1C spliceform containing exon 8 A compared to control neurons. Splicing of CACNA1C is developmentally regulated in the mouse and human with a shift in exon utilization from exon 8 A to 8. These two alternatively spliced exons have been shown to yield channel isoforms with similar electrophysiological features. These findings raise the possibility that decreasing expression of the 8 A isoform of CACNA1C may function as a therapeutic strategy for TS1.

Provided herein are methods, compositions and kits directed to the treatment of Timothy Syndrome.

### SUMMARY

Provided herein are methods of treating an individual having Timothy syndrome, the methods including administering an effective dose of an agent to the individual, wherein the agent modulates the splicing of an 8 A or an 8 exon of CACNA1C.

The present disclosure also provides a method of screening a candidate agent for activity in treating a condition associated with a defect in gene splicing, the method including: (i) administering the candidate agent to human tissue derived from an individual having the condition associated with the defect in gene splicing, (ii) measuring the expression of an exon that is associated with the condition associated with the defect in gene splicing in the human tissue, (iii) comparing the expression of the exon that is associated with the condition associated with the defect in gene splicing in the human tissue in the absence of the administration of the candidate agent.

Compositions and kits for practicing the subject methods are provided.

These and other objects, advantages, and features of the invention will become apparent to those persons skilled in the art upon reading the details of the subject methods and compositions as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1E. The TS mutation enhances splicing of the 8 A CACNA1C spliceform. A. Schematics illustrating the genetic basis of TS (left half) and the resulting channel dysfunction (right half). The heterozygous G>A mutation (black arrow) was located at the 3′ of the exon 8 A. From top to bottom SEQ ID NO: 21-22. B. Control and TS hiPS cells derived from individuals were differentiated to hCO in parallel. C. Schematic of RFLP assay. Left panel, PCR products amplified from the cDNA of hCO consist of 3 potential amplicons. The exon 8-containing amplicon can be recognized by restriction enzyme BamHI. The exon 8 A, exon 8 and exon 7-exon9 amplicons had different sizes on an agarose gel Right panel, RFLP gel image of control and TS samples from day 30, day 60 and day 90 hCO. D. Next-generation sequencing of amplicons generated from day 60 hCO. PCR products were obtained using forward primer targeting exon 7 and reverse primer targeting exon 9. Both primers have an Illumina adapter at their 5′. Proportion of exon 8 A WT, exon 8 A TS, exon 8 and exon 7-9 (n=3 for WT hCOs, n=3 for TS hCO). One-way ANOVA with multiple comparison among groups (for control hCO, F_{2,6}=3.246, P=0.1108; for TS hCO, F_{3,8}=50.28, P<0.0001. ****P<0.0001, ***P<0.001, **P<0.01). E. Generation of mini splicing reporters of TS. Left panel, schematics of experimental procedures for testing the mini splicing reporters. Right panel, DNA fragment of CACNA1C from TS iPS cells were inserted into the backbone of pDup4-1, resulting two vectors, pDup8-8 A-WT and the pDup8-8 A-TS. Different clones of the vectors were used for HEK cell transfection. RNA was extracted and reverse transcribed at 3 days post transfection. cDNA was amplified and loaded on a 2% agarose gel.

FIGS. 2A-2F. Screening of ASOs targeting exon 8 A of CACNA1C. A. Schematic of the ASO design. Red arrow depicts the location of the TS variant. SEQ ID NO: 23 (equivalent to 539038-539158 of NG_008801.2 RefSeqGene) B. qPCR of exon 8 A and exon 8 of ASO-treated dissociated TS hCO. hCO from 2 TS lines (#9-2 and #8-3) were dissociated and plated. Data are mean±SEM. For each of the 2 TS line, 10 μM of each ASO were added to two separated wells, resulting a total of 4 data points for each ASO treatment. Data are mean±SEM. The RNA extraction of the samples was done at 3 days post treatment. Statistics was not performed. C. qPCR analysis of exon 8 A and exon 8 of ASO-treated 3D hCO. Data are mean±SEM. Three TS lines were used (n=3) one-way ANOVA with multiple comparisons between groups, F_{6,13}=8.066, P=0.0009, *P<0.05, **P<0.005. D. RFLP analysis of samples corresponding to those in FIG. 2c. Size of corresponding amplicons were annotated (black triangle) E. A series of concentration of ASO.14 were used to evaluate the dose-dependent splicing modulation on CACNA1C in hCO. Left and right, treatment was done at differentiation day 30 (n=3) and day 90 (n=4), respectively. one-way ANOVA with multiple comparisons between groups was used (Day 30, F_{5,12}=5.131, P=0.0095. Day 90, F_{5,18}=36.81, P<0.0001, *P<0.05, ****P<0.0001). F. Flow cytometry of hCO after 3 days of incubation with 1 μM Cy5-ASO.14. hCO were dissociated and stained with cell surface protein CD90. Non-treated hCO serves as a control for gating Cy5 and CD90.

FIGS. 3A-3F. ASO restored the channel function of Caᵥ1.2 in TS neurons. A. Experimental design for evaluating the rescuing effects of ASOs. B. Representative traces of calcium signal Fura-2, AM C. Comparison of residual Ca2+ measured by Fura-2, AM in ASO-treated neurons. (Left: data pooled across hiPS cell lines; right: data separated by cell line. Each dot represents a cell (n=2017 cells); Kruskal-Wallis test, P<0.0001. Ctrl vs. TS, ****P<0.0001; TS vs. ASO.14, ***P<0.001; TS vs. ASO.17, P<0.0001; TS vs. ASO.18, ****P<0.0001.) D. Representative example of patch clamp recordings from AAV-SYN1-eYFP infected neurons. E. Representative examples of barium currents with voltage steps of depolarization (5 seconds) from −70 mV to −25 mV, −15 mV, −5 mV, respectively. F. Summary graph of barium current inactivation (percentage of inactivated current compared with amplitude of peak current) at 2 seconds for the maximal current in each condition. Ctrl Scr, n=15 cells from 2 lines, TS Scr, n=22 cells from 2 lines, TS ASO 17, n=14 cells from 2 lines, TS ASO 14, n=10 cells from 1 line. one-way ANOVA with multiple comparisons between groups was used (F_{3,57}=25.92, P<0.0001, ****P<0.0001)

FIGS. 4A-4D. ASOs partially rescued the interneuron migration defects in the hFAs. A. Schematic of evaluating the ASO effects on interneuron migration of TS. Prior to the fusion of hSO and hCO, i.e., the assembly of hFAs, the hSO were infected with lentiviral vectors encoding the eGFP driven by the mouse Dl×1/2 enhancer for labelling interneurons. After around 4 weeks of fusion, live imaging was taken on the actively migrating eGFP+ interneurons in the hCO, establishing a baseline prior to ASO treatment. After 2 weeks of incubation of the ASOs and the hFAs, the migration was measured again for evaluating ASO effects. B. Saltation frequencies were calculated by the number of saltation divided by the period of imaging time. Images were taken every 20 min over a total imaging time of around 15 h on a confocal microscope (Leica Stellaris). For b and c, 13 control cells and 16 TS cells were used for analyzing the baseline prior to ASO treatment. For post ASO treatment, 30 cells of control ASO.Scr group, 37 cells of TS ASO.Scr group, 38 cells of ASO.14 group and 26 cells of ASO.17 group were used. one-way ANOVA with multiple comparisons between groups was used for the 4 ASO-treated groups (F_{3,127}=15.35, P<0.0001, ***P<0.0001). Unpaired t test was used to compare the baseline control and TS groups (P<0.0001). C. Average saltation length was calculated by the total migrating length divided by the saltation number, reflecting the average length of each saltation of the interneurons. one-way ANOVA with multiple comparisons between groups was used for the 4 ASO-treated groups (F_{3,120}=5.468, P=0.0015, *P<0.05). Unpaired t test was used to compare the baseline control and TS groups (*P<0.05). D. Representative images of the saltatory movement (yellow triangles) of Dl×1/2b:eGFP+ interneurons.

FIGS. 5A-5I. In vivo testing of ASO in rats transplanted with hCO. A. Schematics of the hCO transplantation. The transplanted hCO (t-hCO) B. Representative MRI of rats with t-hCO used in this study (scale bar 4 mm). C. Immunostaining of the human-specific HNA of the t-hCO. D. Left panel, qPCR of the exon 8 A and exon 8 of rat Cancan1c from rats bearing t-hCO and received injection of 300 μg ASO.14 dissolved in 30 μl PBS or PBS only. Samples were collected between 7-9 days post injection. The primers of Gapdh, exon 8 A and exon 8 of rat Cacna1c are specific for rats. Rat cortex exon8 A (P<0.01, ASO vs. PBS, two-tailed Student's t-tests), cerebellum exon8 A (P<0.05 two-tailed Student's t-tests). Right panel, qPCR of the exon 8 A and exon 8 of human CACNA1C from t-hCO obtained from the cortex of rats. The primers for GAPDH, exon 8 A and exon 8 of CACNA1C in this dataset are specific for human to avoid interference of unspecific amplification of rat transcripts. t-hCO (ASO vs. PBS, P=0.0005, two-tailed Student's t-test) E. Calcium imaging of t-hCO obtained from ASO-treated transplanted rats. The slices of t-hCO were prepared along with the collection for RNA shown in FIGS. 5E and 5F (scale bar, 100 μm). Slices of t-hCO were incubated with Calbryte 520 AM for an hour and then imaged under confocal microscope for around 30 seconds for baseline calcium levels in a low KCl solution, after which the low KCl solution was immediately replaced with 67 mM KCl solution. Live imaging was continuously taken for a total period of 25 minutes (scale bar, 100 μm). F. Representative traces of calcium signal measured by Calbryte 520 AM. G. Residual Ca²⁺ measured by Calbryte 520, AM. t-hCO samples were collected from ASO- and PBS-injected rats shown in d. H. Representative images of cell morphology tracing using Golgi staining. I. Sholl analysis of control, TS and TS+ASO neurons in t-hCO. (n=24 control t-hCO neurons, n=24 TS t-CO neurons, n=11 TS+ASO t-hCO neurons). Data are presented as mean±SEM.

FIGS. 6A-6E. The TS variant caused abnormal splicing events. A. qPCR analysis of the exon 8 A and exon 8 of CACNA1C from samples collected at different days of hCO. Data are mean±SEM. Each dot represents an individual sample collected from different differentiations. Left, exon 8 A (P<0.05 TS vs Ctrl, day 60, P<0.0001 TS vs. Ctrl, day 90, two-tailed Student's t-tests). Right, exon 8 (P<0.05, two-tailed Student's t-tests). B. Next generation sequencing of the amplicons generated from day 60 hCO. The PCR products were obtained using forward primer targeting exon 7 and reverse primer targeting exon 9, both primers have an illumine adapter at their 5′. C. Sequencing conformation of the minisplicing reporter vectors. The G-to-A TS mutation was annotated (red). From top to bottom SEQ ID NOs 24-30. D. Schematics illustrating the splicing outcomes from the pDup8-8 A-WT vs. pDup8-8 A-TS. E. Sequencing of the amplicons generated from the cDNA of transfected HEK cells. n=4, one-way ANOVA with multiple comparisons between groups was used (F_{7,24}=2295, P<0.0001, ****P<0.0001).

FIGS. 7A-7D. ASOs modulated CACAN1C splicing. A. Amplicon sequencing of the ASO-treated hCO. Samples were collected after 3 days of ASO treatment (concentration 10 μM). Left panel, summary of the average percentages of the PCR product composition. Right panel, data from the left panel separated by TS lines. B. RFLP analysis of ASOs-treated hCO for 15 days (upper panel) and 30 days (lower panel). 10 μM ASO were added into hCO, after which culture media containing no ASO was changed every 3-4 days till reaching 15 days and 30 days of ASO incubation. C. qPCR analysis of hCO receiving a single dose of ASO for 30 days (n=3, one-way ANOVA with multiple comparisons between groups was used, F_{4,10}=18.59, P=0.0001, ***P<0.0001.) D. 1 μM ASO.14 was incubated with hCO for a period of from 1 h. to 72 h. qPCR analysis of exon 8 A (left) and exon 8 (right.) n=3, one-way ANOVA with multiple comparisons between groups was used. (F_{5,12}=18.96, ****P<0.0001, ***P=0.0001, *P=0.0306)

FIGS. 8A-8C. Western blot of human CaV1.2 of ASO-treated hCO. A. Western blot of hCO treated with ASO.14, ASO.17, ASO.18 or ASO.Scr. B. CaV1.2 densities normalized to internal GAPDH densities were plotted. No statistical difference was found among groups, one-way ANOVA with multiple comparisons were used (F_{4,31}=0.3548, P=0.8387.). Each dot represents an individual sample from an independent experiment. C. Raw images of the Western blot were corresponding to a and b were shown.

FIGS. 9A-9B. ASOs entered hCO indicated by Cy5 labeled ASO. A. Cy5-ASO fluorescence and immunostaining of CTIP2 of Cy5-ASO.14 treated monolayer cells. hCO were dissociated and plated on glass coverslips. Indicated concentration of Cy5-ASO.14 were added to the monolayer culture for 3 days and fixed for immunostaining. Cy5 fluorescence was directly detected under confocal microscopy. B. qPCR analysis of the exon 8 A of hCO that were incubated with indicated ASOs at 1 μM and were collected after 3 days. n=3 one-way ANOVA with multiple comparisons among groups was used. (***P<0.001, **P<0.01).

FIGS. 10A-10C. Patch clamp of ASO-treated TS and control neurons. A. I-V curves of barium current amplitudes recorded from cortical neurons. I-V curves were fitted with Boltzmann exponential functions. B. Upper panel, representative traces of barium currents in pre-pulse depolarization to −100 mV, −70 mV, −20 mV and 0 mV (3 seconds) followed by test pulse depolarization to 0 mV (3 seconds). Lower panel, representative traces of barium currents in the test pulse depolarizations after pre-pulse depolarization (as indicated in the figure). C. Voltage dependence of barium current inactivation with a test pulse to 0 mV after a series of pre-pulses from −110 mV to +40 mV with an increment of 10 mV. Voltage-dependent inactivation curves of Ctrl and TS+ASO were fitted with exponential functions. (Ctrl scr, n=14 cells from 2 lines, TS scr, n=15 cells from 2 lines TS ASO 17, n=14 cells from 2 lines (ASO 17: n=8 cells; ASO 14, n=6 cells)

FIGS. 11A-11C. Evaluating ASO delivery and effectiveness in rats that contain no t-hCO. A. Sequence alignment between the Cacna1c exon 8 A of rat and human CACNA1C exon 8 A. Red boxes indicated mismatch. ASO.14 hybridize to both Rat Cacna1c exon 8 A and human CACNA1C exon 8 A. From top to bottom SEQ ID Nos 14, 14, and 31-32. B. 80 μg ASO.14 was administrated into Cisterna magna. Rat brain, cerebellum and spinal cord were collected 5 days post injection. C. qPCR analysis of rat Cacna1c exon 8 A and exon 8 after ASO injection (b). 2 rats were used for each condition. For each sample, 2 pieces of adjacent tissue were collected in each brain region (cortex, cerebellum and spinal cord). Statistics was not performed in this experiment.

FIGS. 12A-12G. Effect of in vivo administration of an ASO on dendrite morphology in TS. A. RFLP analysis of t-hCO samples following ASO injection in vivo. Upper panel: n=3 control, n=4 TS, n=4 for TS+ASO t-hCO. Lower panel: n=3 t-hCO per group. The two gel columns for each conditions represent two 2 pieces cut of a t-hCO from the same rat. The same t-hCO were used for qPCR shown in FIG. 5D. B. Western blot of t-hCO treated with ASO.14 or PBS. Data are presented as mean±s.e.m. One-way ANOVA with multiple comparisons among groups. F2, 6=0.07231, P=0.9310. C. Representative images of cell morphology tracing using Golgi staining. D. Analysis of area under the curve (n=24 control t-hCO neurons, n=24 TS t-CO neurons, n=11 TS+ASO t-hCO neurons). Data are presented as mean±s.e.m. One-way ANOVA with multiple comparisons among groups, F2,56=8.134, P=0.0008; ***P=0.0008. E. Quantification of intersection peak radius (n=24 control t-hCO neurons, n=24 TS t-CO neurons, n=11 TS+ASO t-hCO neurons). Data are presented as mean±s.e.m. One-way ANOVA with multiple comparisons among groups. One-way ANOVA with multiple comparisons among groups, F2,56=8.225, P=0.0007; ***P=0.0009. F. Comparison of the longest dendrite length among groups (n=24 control t-hCO neurons, n=24 TS t-CO neurons, n=11 TS ASO t-hCO neurons). Data are presented as mean±s.e.m. One-way ANOVA with multiple comparisons among groups, F2,56=3.266, P=0.0455; *P=0.0257. G. Comparison of total dendrite length among groups (n=24 control t-hCO neurons, n=24 TS t-CO neurons, n=11 TS+ASO t-hCO neurons). Data are presented as mean±s.e.m. One-way ANOVA with multiple comparisons among groups, F2,54=7.873, P=0.0010; **P=0.0011.

The invention is best understood from the following detailed description when read in conjunction with the accompanying drawings.

### Definitions

Before describing exemplary embodiments in greater detail, the following definitions are set forth to illustrate and define the meaning and scope of the terms used in the description.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It must be noted that as used herein and in the appended claims, the singular forms “a”, “an”, and “the” include plural referents unless the context clearly dictates otherwise. For example, the term “an agent” refers to one or more agents, i.e., a single agent and multiple agents. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer containing purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

By “hybridizable” or “complementary” or “substantially complementary” it is meant that a nucleic acid (e.g. RNA, DNA) has a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). In addition, for hybridization between two RNA molecules (e.g., dsRNA), and for hybridization of a DNA molecule with an RNA molecule (e.g., when a DNA target nucleic acid base pairs with a sensor RNA, etc.): G can also base pair with U. For example, G/U base-pairing is partially responsible for the degeneracy (i.e., redundancy) of the genetic code in the context of tRNA anti-codon base-pairing with codons in mRNA. Thus, in the context of this disclosure, a G (e.g., of a protein-binding segment (e.g., dsRNA duplex) of a sensor RNA molecule; of a target nucleic acid (e.g., target DNA) base pairing with a guide RNA) is considered complementary to both a U and to C. For example, when a G/U base-pair can be made at a given nucleotide position of a protein-binding segment (e.g., dsRNA duplex) of a sensor RNA molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more).

It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can include 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The term “neural organoid” as used herein refers to a range of brain-region specific organoids. Neural organoids encompass any organoid that contains neurons from any part of the brain. Cortical organoids, midbrain organoids, striatal organoids, spinal cord/hindbrain, ventral forebrain organoids, and organoids having any combination of the aforementioned organoids are encompassed by the term neural organoids. The terms “organoid” and “spheroid” may be used interchangeably.

The terms “anatomical integration” or “anatomically integrated” as used herein refer to neural tissue that is innervated by host neurons. The human neural tissue present within the non-human mammalian animal model has neurons originating from the non-human mammalian animal model nervous system and thus the human neural tissue that is anatomically integrated into the non-human mammalian animal model has both human neural tissue and non-human mammalian tissue.

Timothy syndrome (TS) is characterized by multiorgan dysfunction, including severe arrhythmias, webbing of fingers and toes, congenital heart disease, immune deficiency, intermittent hypoglycemia, cognitive abnormalities, epilepsy and ASD. There are two recognized types of Timothy syndrome, classical (type-1) and atypical (type-2). They are both caused by mutations in CACNA1C, the gene encoding the calcium channel Caᵥ1.2α subunit. Timothy syndrome mutations in CACNA1C cause delayed channel closing, thus increased intracellular calcium. These mutations are in exon 8 (atypical form) and exon 8 A (classical form), an alternatively spliced exon. Exon 8 A is highly expressed in the heart, brain, gastrointestinal system, lungs, immune system, and smooth muscle. Exon 8 A is also expressed early in development. Exon 8 is also expressed in these regions and its level is roughly five-fold higher than exon 8 A expression. The genomic loci of CACNA1C is 87465-732164 of NG_008801.2 (RefSeqGene Number). CACNA1C has multiple splice variants having the following accession numbers: NM_000719.7, NM_001129827.2, NM_001129829.2, NM_001129830.3, NM_001129831.2, NM_001129832.2, NM_001129833.2, NM_001129834.2, NM_001129835.2, NM_001129836.2, NM_001129837.2, NM_001129838.2, NM_001129839.2, NM_001129840.2, NM_001129841.2, NM_001129842.2, NM_001129843.2, NM_001129844.2, NM_001129846.2, NM_001167623.2, NM_001167624.3, NM_001167625.2, and NM_199460.4. An exemplary accession number containing exon 8 A is NM_001167625.2. An exemplary accession number containing exon 8 is NM_000719.7. Mutations and amino acid substitutions are known in the art and have been described by, for example, Bauer et al. (Front Pediatr. 2021 May 17:9:66854), Han et al. (Exp Biol Med (Maywood). 2019 September; 244(12): 960-971), Splawski et al. (Cell 2004 Oct. 1; 119(1):19-31) and Splawski et al. (Proc Natl Acad Sci USA 2005 Jun. 7; 102(23):8089-96) each of which is specifically incorporated by reference herein.

The terms “astrocytic cell,” “astrocyte,” etc. encompass cells of the astrocyte lineage, i.e., glial progenitor cells, astrocyte precursor cells, and mature astrocytes, which for the purposes of the present invention arise from a non-astrocytic cells (i.e., glial progenitors). Astrocytes can be identified by markers specific for cells of the astrocyte lineage, e.g. GFAP, ALDH1 L1, AQP4, EAAT1 and EAAT2, etc. Markers of reactive astrocytes include S100, VIM, LCN2, FGFR3 and the like. Astrocytes may have characteristics of functional astrocytes, that is, they may have the capacity of promoting synaptogenesis in primary neuronal cultures; of accumulating glycogen granules in processes; of phagocytosing synapses; and the like. A “astrocyte precursor” is defined as a cell that is capable of giving rise to progeny that include astrocytes.

Astrocytes are the most numerous and diverse neuroglial cells in the CNS. An archetypal morphological feature of astrocytes is their expression of intermediate filaments, which form the cytoskeleton. The main types of astroglial intermediate filament proteins are glial fibrillary acidic protein (GFAP) and vimentin; expression of GFAP, ALDH1L1 and/or AQP4P are commonly used as a specific marker for the identification of astrocytes.

The terms “oligodendrocyte,” “oligodendrocyte progenitor cell,” etc. can encompass cells of the oligodendrocyte lineage, i.e., neural progenitor cells that ultimately give rise to oligodendrocytes, oligodendrocyte precursor cells, and mature and myelinating oligodendrocytes, which for the purposes of the present invention arise from a non-oligodendrocyte cell by experimental manipulation. Oligodendrocytes may have functional characteristics, that is, they may have the capacity of myelinating neurons; and the like. An “oligodendrocyte precursor” or “oligodendrocyte progenitor cell” is defined as a cell that is capable of giving rise to progeny that include oligodendrocytes. Oligodendrocytes may be present in the assembloids.

Oligodendrocytes are the myelin-forming cells of the central nervous system. An oligodendrocyte extends many processes which contact and repeatedly envelope stretches of axons. Subsequent condensation of these wrapped layers of oligodendrocyte membrane form the myelin sheath. One axon may contain myelin segments from many different oligodendrocytes.

Calcium sensors. Neural activity causes rapid changes in intracellular free calcium, which can be used to track the activity of neuronal populations. Art-recognized sensors for this purpose include fluorescent proteins that fluoresce in the presence of changes in calcium concentrations. These proteins can be introduced into cells, e.g., hiPSC, by including the coding sequence on a suitable expression vector, e.g., a viral vector, to genetically modify neurons generated by the methods described herein. GCaMPs are widely used protein calcium sensors, which contain a fluorescent protein, e.g., GFP, the calcium-binding protein calmodulin (CaM), and CaM-interacting M13 peptide, although a variety of other sensors are also available. Many different proteins are available, including, for example, those described in Barreto-Chang et al. (2009, J Vis Exp, doi:10.3791/1067), Zhao et al. (2011) Science 333:1888-1891; Mank et al. (2008) Nat. Methods 5(9):805-11; Akerboom et al. (2012) J. Neurosci. 32(40):13819-40; Chen et al. (2013) Nature 499(7458):295-300; etc.; and as described in U.S. Pat. Nos. 8,629,256, 9,518,980 and 9,488,642 and 9,945,844 which are specifically incorporated by reference herein.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” are used interchangeably herein and refer to any mammalian subject for whom diagnosis, treatment, or therapy is desired, particularly humans.

### DETAILED DESCRIPTION

Before the present compositions and methods are described, it is to be understood that this invention is not limited to particular compositions and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a reprogramming factor polypeptide” includes a plurality of such polypeptides, and reference to “the induced pluripotent stem cells” includes reference to one or more induced pluripotent stem cells and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Methods for Treating an Individual Having Timothy Syndrome

As summarized above, methods are provided for treating an individual having Timothy syndrome. Aspects of the methods include administering an effective dose of an agent to the individual to treat the individual having Timothy syndrome, wherein the agent modulates the splicing of an 8 A or an 8 exon of CACNA1C.

Timothy syndrome may refer to Timothy syndrome type 1 or Timothy syndrome type 2. In some embodiments, the individual being treated for Timothy syndrome is being treated for Timothy syndrome type 1. In some embodiments, the individual being treated for Timothy syndrome type 1 has a mutation in the 8 A exon of CACNA1C that results in a G406R amino acid substitution in the Caᵥ1.2α subunit. In some embodiments, the individual being treated for Timothy syndrome is being treated for Timothy syndrome type 2. In some embodiments, the individual being treated for Timothy syndrome type 2 has a mutation in the 8 exon of CACNA1C that results in a G406R amino acid substitution in the Caᵥ1.2α subunit. In some embodiments, the individual being treated for Timothy syndrome type 2 has a mutation in the 8 exon of CACNA1C that results in a G402S amino acid substitution in the Caᵥ1.2α subunit.

Prior to the administration of the agent, the individual may be screened to determine if the individual would receive benefit from the treatments disclosed herein. Individuals that may receive particular benefit include, without limitation, individuals that have a mutation in the 8 A exon of CACNA1C that results in a G406R amino acid substitution in the Caᵥ1.2α subunit, individuals that have a mutation in the 8 exon of CACNA1C that results in a G406R amino acid substitution in the Caᵥ1.2α subunit, individuals that have a mutation in the 8 exon of CACNA1C that results in a G402S amino acid substitution in the Caᵥ1.2α subunit, etc. In some embodiments, the mutation in the 8 A exon of CACNA1C that results in a G406R amino acid substitution in the Caᵥ1.2α subunit is a c.1216G>A mutation in exon 8 A. In some embodiments, the mutation in the 8 exon of CACNA1C that results in a G406R amino acid substitution in the Caᵥ1.2α subunit is a c.1216G>A mutation in exon 8. In some embodiments, the mutation in the 8 exon of CACNA1C that results in a G402S amino acid substitution in the Caᵥ1.2α subunit is a c.1204G>A mutation in exon 8. In some embodiments, the c.1216G>A mutation in exon 8 A is referred to as rs79891110. In some embodiments, the c.1216G>A mutation in exon 8 is referred to as rs786205745. In some embodiments, the c.1204G>A mutation in exon 8 is referred to as rs587782933. The c.1216G>A mutation in exon 8 A, the c.1216G>A mutation in exon 8, and the c.1204G>A mutation in exon 8 are known in the art and have been described by, for example, Splawski et al. (Cell 2004 Oct. 1; 119(1):19-31) and Splawski et al. (Proc Natl Acad Sci USA 2005 Jun. 7; 102(23):8089-96).

The screening of the individuals generally involves genetic testing of the individual. In some embodiments, the genetic testing is genotyping assay. In some embodiments, the genetic testing is a restriction fragment length polymorphism (RFLP) assay. In some instances, the genotyping assay includes genotyping a CACNA1C gene. In some instances, the genotyping involves assaying for the presence of a polymorphism or mutation. The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene”. A specific genetic sequence at a polymorphic region of a gene is an allele. A polymorphic region can be a single nucleotide, the identity of which differs in different alleles. A polymorphic region can also be several nucleotides long. In some instances, the genotyping includes assessing a single nucleotide polymorphism (SNP), including assay 2 or more SNPs. While the SNPs may vary, as desired, in some instances the SNPs are CACNA1C SNPs, such as but not limited to: rs79891110, rs786205745, or rs587782933. The SNPs can be identified by detecting any of the component alleles using any of a variety of available techniques, including: 1) performing a hybridization reaction between a nucleic acid sample and a probe that is capable of hybridizing to the allele; 2) sequencing at least a portion of the allele; or 3) determining the electrophoretic mobility of the allele or fragments thereof (e.g., fragments generated by endonuclease digestion such as a RFLP assay). The allele can optionally be subjected to an amplification step prior to performance of the detection step. Amplification methods that may be employed include those selected from the group consisting of: the polymerase chain reaction (PCR), the ligase chain reaction (LCR), strand displacement amplification (SDA), cloning, and variations of the above (e.g. RT-PCR and allele specific amplification). Oligonucleotides necessary for amplification may be selected, for example, from within the CACNA1C gene loci, either flanking the exon of interest (e.g., exon 8 and/or exon 8 A; as required for PCR amplification) or directly overlapping the marker (as in ASO hybridization). In some embodiments, the sample is hybridized with a set of primers, which hybridize 5′ and 3′ in a sense or antisense sequence to the disease associated allele, and is subjected to a PCR amplification. Exemplary primers for exon 8 of the CACNA1C gene are Forward primer CTTCTTTCCTAACTTTCCTTCG (SEQ ID NO: 33) and reverse primer CTGCGTTGTGGAGAGGACATA (SEQ ID NO: 34). Exemplary primers for exon 8 A of the CACNA1C gene are Forward primer GTGCCTCACTAACTATCATTCC (SEQ ID NO: 35) and reverse primer AAATCAAGACCTTTTTCCTTGGT (SEQ ID NO: 36). In some embodiments, an allelic discrimination assay such as a TaqMan SNP genotyping assay, may be used, e.g., as described in 15he L-K, Lee T-Y, Tan JAMA et al. (2015); Int J Lab Hematol 37(1):79-89 which is specifically incorporated by reference herein.

The screening may be performed at a time in which the individual would receive timely intervention with the treatments disclosed herein. In some embodiments, the screening is performed when the individual is in utero, i.e., before birth. In some embodiments, the screening is performed when the individual is an infant. In some embodiments, the screening is performed when the individual is a toddler. In some embodiments, the screening is performed when the individual is in early childhood. In some embodiments, the screening is performed when the individual is in late childhood. In some embodiments, the screening is performed when the individual is an adolescent. In some embodiments, the screening is performed when the individual is an adult. Infancy as used herein refers to an individual that is a neonate and up to one year of age. Toddler as used herein refers to an individual who is one to five years old. Early childhood as used herein refers to an individual who is three to eight years old. Late childhood as used herein refers to an individual who is nine to eleven years old. Adolescent as used herein refers to an individual who is twelve to eighteen years old.

The agents of the present disclosure are administered to an individual having, or at least suspected of having (such as by screening, e.g., as described above) Timothy syndrome, such that the individual may be an individual diagnosed as having Timothy syndrome. The agents may be administered using any method deemed useful for the treatment of Timothy syndrome. In some embodiments, the agent is administered systemically. In some embodiments, the systemic administration is through subcutaneous, intravenous, intracisternal magna, or intrathecal administration. In some embodiments, the agent is administered locally. In some embodiments, the local administration is intracerebroventricular, intraparenchymal, or intracardiac administration.

The agents of the present disclosure are administered to an individual in an effective dose. An effective dose may be any dose that achieves the desired therapeutic effect. Therapeutic effects include, without limitation, amelioration of symptoms related to Timothy syndrome, decreased expression of the 8 A exon of CACNA1C, decreased expression of the 8 exon of CACNA1C, etc. An effective dose may be up to about 1 μg/kg body weight, up to about 2.5 μg/kg, up to about 5 μg/kg, up to about 10 μg/kg, up to about 25 μg/kg, up to about 50 μg/kg, up to about 100 μg/kg, up to about 250 μg/kg, up to about 500 μg/kg, up to about 750 μg/kg, up to about 1 mg/kg, up to about 2.5 mg/kg, up to about 5 mg/kg, up to about 10 mg/kg or more, administered as the agent alone or as a composition disclosed herein.

The administration may occur at a range of different intervals. For instance, the agent may be administered once a day, once a week, once every two weeks, once every three weeks, once a month, once every two months, once every three months, once every four months, once every five months, once every six months, once every seven months, once every eight months, once every nine months, once every ten months, once every eleven months, or once a year. The administration may also occur on a variable schedule where a medical professional assesses the individual having Timothy syndrome and determines when an administration is necessary.

The agent may be any agent that modulates the splicing of an 8 A or an 8 exon of CACNA1C. Agents of the present disclosure may be nucleic acids. When the agent is a nucleic acid, the nucleic acid can be any nucleic acid that modulates the splicing of an 8 A or an 8 exon of CACNA1C. Non-limiting examples of nucleic acids of the present disclosure include, without limitation, an antisense oligonucleotide, a double-stranded silencing RNA (siRNA), etc. When the agent is a nucleic acid, the nucleic acid may have a modification to increase the stability or the activity of the nucleic acid. Modifications that find use in the present disclosure include, without limitation, phosphonothioate, a 2′-O-methoxyethylribose, 5-methylcytosine, 7-deaza-dG, locked nucleic acids, etc. When a nucleic acid is modified, every base within the nucleic acid may be modified or a specific proportion of bases may be modified. For instance, 10%, 20%, 30%, 40%, 50%, 60%, 70%, 80%, 90%, or 100% of the bases may be modified. In some embodiments, the bases are modified with a mixture of the modifications described above. In some embodiments, the bases are modified with one type of modification described above. In some embodiments, a portion or all of the bases are modified with 2′-O-methoxyethylribose modifications. In some embodiments, a portion or all of the bases are modified with phosphonothioate modifications. In some embodiments, a portion or all of the bases are modified with 5-methylcytosine modifications. In some embodiments, a portion of the bases are modified with 7-deaza-dG modifications. In some embodiments, a portion or all of the bases are modified with locked nucleic acids modifications.

The length of the nucleic acids of the present disclosure may be any length that is beneficial to the activity or function of the nucleic acid. For instance, the nucleic acid may be 15 to 35 nucleotides in length. The nucleic acid may be any intervening length including, without limitation, 15, 16, 17, 18, 19, 20, 21, 22, 23, 24, 25, 26, 27, 28, 29, 30, 31, 32, 33, 34, or 35 nucleotides in length.

The nucleic acids of the present disclosure may contain a particular nucleic acid sequence. Particular nucleic acid sequences that find use in the present disclosure may be any nucleic acid sequence that modulates the splicing of an 8 A or an 8 exon of CACNA1C. When an individual is being treated for Timothy syndrome type 1, the nucleic acid sequences may contain AAATAGATCCAGGGCCAGTC (SEQ ID NO: 14), CAGTCCCTTCCTACGGCATC (SEQ ID NO: 15), or CCTTCCTACGGCATCATTGA (SEQ ID NO: 16). In some embodiments, the nucleic acid contains AAATAGATCCAGGGCCAGTC (SEQ ID NO: 14).

The agents of the present disclosure may have a range of activities. The activities of the agents of the present disclosure include, without limitation, decreased expression of the 8 A exon of CACNA1C, decreased expression of the 8 exon of CACNA1C, reduction of residual CA²⁺ levels in a neuron following neuronal depolarization, amelioration of symptoms related to Timothy syndrome, etc. In some embodiments, the agents of the present disclosure provide a therapeutic effect in the absence of reducing total Caᵥ1.2 protein amounts.

When the expression of the 8 A or 8 exon is decreased, the exon may be decreased by a range of different amounts. For instance, the expression of the 8 A or 8 exon may be decreased by at least about 2 fold, 5 fold, 10 fold, 20 fold, 30 fold, 40 fold, 50 fold, 100 fold, 200 fold, 300 fold, 500 fold, 1000 fold, 10000 fold or more than 10000 fold relative to the level of expression of the 8 A or 8 exon of CACNA1C prior to administration of the agent.

Ca²⁺ levels in neurons following the depolarization of the neuron are elevated in individuals having Timothy syndrome relative to individuals who do not have Timothy syndrome. In some embodiments, the agents disclosed herein reduce the Ca²⁺ levels in a neuron following neuronal depolarization. The reduction in Ca²⁺ levels may be 0.05 or greater, such as 0.10 or greater, 0.15 or greater, 0.20 or greater, 0.25 or greater, or 0.30 or greater as measured using calcium imaging such as the methods described below (e.g., in Example 1) or in Barreto-Chang et al. (2009, J Vis Exp, doi:10.3791/1067). In some embodiments, the method of calcium imaging involves calculating a residual calcium level using the formula (C-A)/(B-A) where A is the baseline value, B is the peak value following depolarization and C is the decay value. In some embodiments, the Ca²⁺ levels in a neuron in an individual having Timothy syndrome following neuronal depolarization are reduced to the levels found in neurons from individuals not having Timothy syndrome.

In some embodiments, the methods of the present disclosure include administering two or more agents. The two or more agents may be administered sequentially or simultaneously. The two or more agents may be the same type of agent or a different type of agent. In some embodiments, the two or more agents are nucleic acids. In some embodiments, the two or more nucleic acids target different parts of an 8 A or 8 exon of CACNA1C. In some embodiments, one of the two or more agents is an agent traditionally used to treat Timothy syndrome. Agents that are used traditionally to treat Timothy syndrome include, without limitation, a beta blocker, mexiletine, etc. When the agent is a beta blocker the beta blocker may be any beta blocker deemed useful. Beta blockers that find use in the present disclose include, without limitation, acebutolol, atenolol, labetalol, pindolol, propranolol, nadolol, timolol, sotalol, bisoprolol, nebivolol, metoprolol, etc.

Methods for Screening a Candidate Agent for Activity in Treating a Condition Associated with a Defect in Gene Splicing

As summarized above, methods are provided for screening a candidate agent for activity in treating a condition associated with a defect in gene splicing, the method including: (i) administering the candidate agent to human tissue derived from an individual having the condition associated with the defect in gene splicing, (ii) measuring the expression of an exon that is associated with the condition associated with the defect in gene splicing in the human tissue, (iii) comparing the expression of the exon that is associated with the condition associated with the defect in gene splicing in the human tissue in the absence of the administration of the candidate agent.

The screening methods disclosed herein are suitable for any condition that is associated with a defect in gene splicing. In some embodiments, the condition may result in impairments and/or symptoms in more than one organ. For instance, the conditions may cause impairments and/or symptoms in both the brain and the heart or the liver and the kidneys or any combination of organs. Conditions associated with defects in gene splicing include, without limitation, spinal muscular atrophy, Taupathies, Hutchinson-Gilford progeria syndrome, hypercholesterolemia, Medium-chain acyl-CoA dehydrogenase Parkinson's disease, Alzheimer's disease, Amyotrophic lateral sclerosis (ALS), Frontotemporal dementia, Familial dysautonomia, Timothy syndrome, etc. Conditions associated with defects in gene splicing are known in the art and have been described in, for example, Tazi et al. (Biochim Biophys Acta. 2009 January; 1792(1):14-26) which is specifically incorporated by reference herein. In some embodiments, the condition is a neuropsychiatric condition. Neuropsychiatric conditions associated with defects in gene splicing include, without limitation, Parkinson's disease, Alzheimer's disease, Amyotrophic lateral sclerosis (ALS), Frontotemporal dementia, Familial dysautonomia, Timothy syndrome, etc. Neuropsychiatric conditions associated with defects in gene splicing are known in the art and have been described in, for example, Li et al. (Transl Neurodegener. 2021 May 20; 10(1):16) which is specifically incorporated by reference herein. In some embodiments, the individual having the neurological condition associated with the defect in gene splicing has a genetic mutation that results in the defect in gene splicing.

The candidate agents of the present disclosure are administered to human tissue derived from an individual having the condition associated with the defect in gene splicing. The candidate agents may be administered using any method deemed useful. In some embodiments, the agent is administered systemically. In some embodiments, the systemic administration is through subcutaneous, intravenous, intracisternal magna, or intrathecal administration. In some embodiments, the agent is administered locally. In some embodiments, the local administration is intracerebroventricular, intramuscular, intrarenal, intra-hepatic, intraparenchymal, or intracardiac administration.

The candidate agent may be any agent that reduces the expression of an exon that is associated with a neurological condition associated with a defect in gene splicing. Candidate agents of the present invention include, without limitation, a nucleic acid, a small molecule, a protein, or a compound. When the candidate agent is a nucleic acid, the nucleic acid includes, without limitation, an antisense oligonucleotide, a double stranded silencing RNA (siRNA), etc.

The human tissue derived from an individual having a condition associated with a defect in gene splicing can be any tissue related to the condition. The human tissue includes, without limitation, gastrointestinal tissue, lung tissue, liver tissue, vascular tissue, retina tissue, neural tissue, etc. In some embodiments, the tissue is in the form of an organoid. Methods of generating organoids of the tissues disclosed above are known in the art and have been described in, for example, Wahlin et al. (Sci Rep. 2017 Apr. 10; 7(1):766), van den Berg et al. (Stem Cell Reports. 2018 Mar. 13; 10(3):751-765), Hu et al. (Cell. 2018 Nov. 29; 175(6):1591-1606.e19), Hild et al. (Curr Protoc Stem Cell Biol. 2016 May 12; 37: IE.9.1-IE.9.15), Salahudeen et al. (Nature. 2020 December; 588(7839):670-675), Barkauskas et al. (J Clin Invest. 2013 Jul; 123(7):3025-36), McCracken (Nature. 2014 Dec. 18; 516(7531):400-4), Spence et al. (Nature. 2011 Feb. 3; 470(7332):105-9), Hofbauer et al. (Cell. 2021 Jun. 10; 184(12):3299-3317.e22), Wimmer et al. (Nature. 2019 January; 565(7740):505-510), Revah et al. Nature. 2022 Oct; 610(7931):319-326 and U.S. Patent application 63/350,367, each specifically incorporated by reference herein.

The measuring of the expression of an exon that is associated with the condition associated with the defect in gene splicing in the human tissue may be done in any way deemed useful. Useful methods for the measurement of exon expression include, without limitation, quantitative polymerase chain reaction (qPCR), Northern blotting, RNA-seq, microarray, fluorescent in situ hybridization, serial analysis of gene expression (SAGE), etc.

Compositions

Also provided are compositions for practicing the methods as described in the present disclosure. In general, subject compositions may have an agent as described above in addition to a pharmaceutically acceptable excipient. In some embodiments, the subject compositions contain a second agent as described above.

The agent may be any agent that inhibits the splicing of an 8 A or an 8 exon of CACNA1C. Agents of the present disclosure may be nucleic acids. When the agent is a nucleic acid, the nucleic acid can be any nucleic acid that inhibits the splicing of an 8 A or an 8 exon of CACNA1C. Non-limiting examples of nucleic acids of the present disclosure include, without limitation, an antisense oligonucleotide, a double stranded silencing RNA (siRNA), etc. When the agent is a nucleic acid, the nucleic acid may have a modification to increase the stability or the activity of the nucleic acid. Modifications that find use in the present disclosure include, without limitation, phosphonothioate, a 2′-O-methoxyethylribose, 5-methylcytosine, 7-deaza-dG, locked nucleic acids, etc. When a nucleic acid is modified, every base within the nucleic acid may be modified or a specific proportion of bases are modified. For instance, 10%, 20%, 30%, 40%, 50%, 60%, 70%, 80%, 90% or 100% of the bases may be modified. In some embodiments, the bases are modified with a mixture of the modifications described above. In some embodiments, the bases are modified with one type of modification described above. In some embodiments, a portion or all of the bases are modified with 2′-O-methoxyethylribose modifications. In some embodiments, a portion or all of the bases are modified with phosphonothioate modifications. In some embodiments, a portion or all of the bases are modified with 5-methylcytosine modifications. In some embodiments, a portion of the bases are modified with 7-deaza-dG modifications. In some embodiments, a portion or all of the bases are modified with locked nucleic acids modifications.

The length of the nucleic acids of the present disclosure may be any length that is beneficial to the activity or function of the nucleic acid. For instance, the nucleic acid may be 15 to 35 nucleotides in length. The nucleic acid may be any intervening length including, without limitation, 15, 16, 17, 18, 19, 20, 21, 22, 23, 24, 25, 26, 27, 28, 29, 30, 31, 32, 33, 34, or 35 nucleotides in length.

The nucleic acids of the present disclosure may contain a particular nucleic acid sequence. Particular nucleic acid sequences that find use in the present disclosure may be any nucleic acid sequence that inhibits the splicing of an 8 A or an 8 exon of CACNA1C. When an individual is being treated for Timothy syndrome type 1, the nucleic acid sequences may contain AAATAGATCCAGGGCCAGTC (SEQ ID NO 14), CAGTCCCTTCCTACGGCATC (SEQ ID NO 15; ASO17), or CCTTCCTACGGCATCATTGA (SEQ ID NO 16). In a preferred embodiment, the nucleic acid contains AAATAGATCCAGGGCCAGTC (SEQ ID NO 14).

In some embodiments, the composition is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the composition may further include a preservative. Suitable preservatives include, but are not limited to, a benzyl alcohol, phenol, chlorobutanol, benzalkonium chloride, and the like. In many cases, the formulation is stored at about 4° C. Pharmaceutical compositions may also be lyophilized, in which case they generally include cryoprotectants such as sucrose, trehalose, lactose, maltose, mannitol, and the like. Lyophilized formulations can be stored over extended periods of time, even at ambient temperatures.

The subject composition may be administered using any method deemed useful for the treatment of Timothy syndrome. In some embodiments, the agent is administered systemically. In some embodiments, the systemic administration is through subcutaneous, intravenous, intracisternal magna, or intrathecal administration. In some embodiments, the agent is administered locally. In some embodiments, the local administration is intracerebroventricular, intraparenchymal, or intracardiac administration.

Each of the active agents can be provided in a unit dose of from about 0.1 μg, 0.5 μg, 1 μg, 5 μg, 10 μg, 50 μg, 100 μg, 500 μg, 1 mg, 5 mg, 10 mg, 50, mg, 100 mg, 250 mg, 500 mg, 750 mg or more.

The composition may be administered in a unit dosage form and may be prepared by any methods well known in the art. Such methods include combining the agent with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar, and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g., PEG, are also good carriers.

Kits

Also, kits for practicing the methods described in the present disclosure are provided. In general, subject kits may contain compositions, e.g., as described above. For instance, the kit may contain one or more agents as described above in addition to a pharmaceutically acceptable excipient. The kit may also include reagents for screening an individual to determine if the individual would receive benefit from the treatments disclosed herein. The reagents may include buffers, enzymes, nucleic acids, proteins, chemicals, small molecules, etc., for genetic testing such as genotyping or RFLP assays.

A subject kit can include any combination of components for performing the methods of the present disclosure. The components of a subject kit can be present as a mixture or can be separate entities. In some cases, components are present as a lyophilized mixture. In some cases, the components are present as a liquid mixture. Components of a subject kit can be in the same or separate containers, in any combination.

The subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a remote site.

### EXPERIMENTAL

Introduction

Human TS1 is a severe genetic disorder caused by the heterozygous c.1216G>A pathogenic variant in exon 8 A of CACNA1C, resulting in a p.G406R missense variant in the α1 subunit of the L-type voltage-gated calcium channel Caᵥ1.2 (7). TS1 impacts multiple organ systems and is one of the most penetrant genetic etiologies of autism spectrum disorder (ASD) and epilepsy (8), and has high mortality (7,9). Variants in CACNA1C have also been strongly associated with other neuropsychiatric disorders, including schizophrenia, bipolar disorder, and attention deficit hyperactivity disorder (10), suggesting that Caᵥ1.2 is a key susceptibility factor for neuropsychiatric conditions.

Studies in hiPS cell-derived cardiomyocytes and neurons in 2D and 3D systems revealed that cells derived from individuals with TS1 have delayed voltage-gated channel inactivation and increased depolarization-induced calcium entry (2,3,11), which leads to abnormal excitability. Moreover, using human forebrain assembloids (hFA) generated by integrating human cortical organoids (hCO) and human subpallial organoids (hSO), defects in cortical interneuron migration were previously described (3). Specifically, it was discovered that TS1 interneurons undergo more frequent nucleokinetic saltations driven by enhanced GABA sensitivity, but the saltation length is reduced due to aberrant cytoskeleton function, leading to overall defective migration (4).

Surprisingly, TS1-derived neurons have abnormally high level of the CACNA1C spliceform containing exon 8 A compared to control neurons (2). Splicing of CACNA1C is developmentally regulated in the mouse and human with a shift in exon utilization from exon 8 A to 8. These two alternatively spliced exons have been shown to yield channel isoforms with similar electrophysiological features (12,13). These findings raise the possibility that decreasing expression of the 8 A isoform of CACNA1C may function as a therapeutic strategy for TS1.

In this study, an antisense oligonucleotide (ASO)-based intervention was developed to effectively decrease exon 8 A splicing in neural cells derived from three TS1 patients. ASOs are short oligonucleotides that can bind to the target RNA, activate cytoplasmic degradation of the target RNAs or modulate the splicing of pre-mRNA inside the nucleus (14), and several ASOs have been successfully implemented in the clinic (15). Here, it was first demonstrated that the TS1 G406R mutation directly enhanced the splicing of the mutated exon 8 A allele. A screen was then performed to identify two ASOs that can, in a time- and dose-dependent way, robustly inhibit splicing of the exon 8 A. Direct application of these two ASO to human cortical neurons in 2D or 3D cultures derived from TS1 patients rescues delayed channel inactivation and the defect in depolarization-induced calcium rise. Moreover, these ASOs restore previously identified cortical interneuron migration defects in forebrain assembloids. Lastly, to verify the ASO effectiveness in an in vivo setting a recent transplantation model was leveraged (21). In this system, human stem cell-derived cortical organoids transplanted (t-CO) into the somatosensory cortex of newborn athymic rats grow to develop mature cell types that integrate into sensory and motivation-related circuits. It was discovered that intrathecal injection of ASO into rats transplanted with human TS1 cortical organoids (t-hCO) results in a robust downregulation of exon 8 A and is followed by rescue of depolarization-induced calcium defects. Taken together, these experiments demonstrate a novel genetic rescue strategy for a devastating neurodevelopmental disorder.

Results

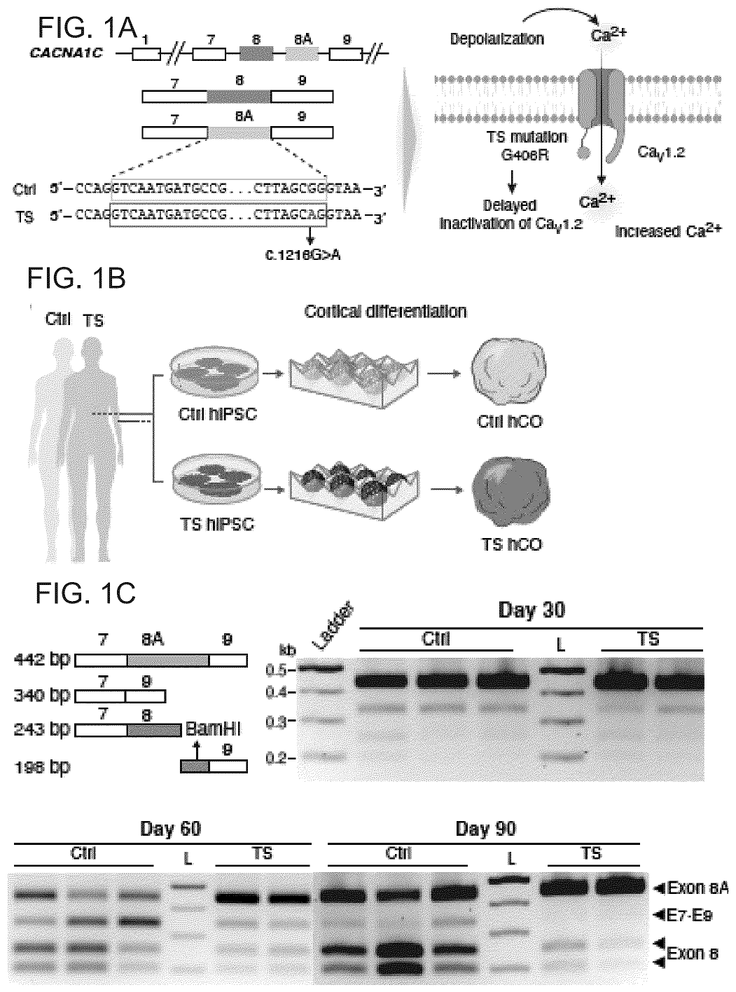

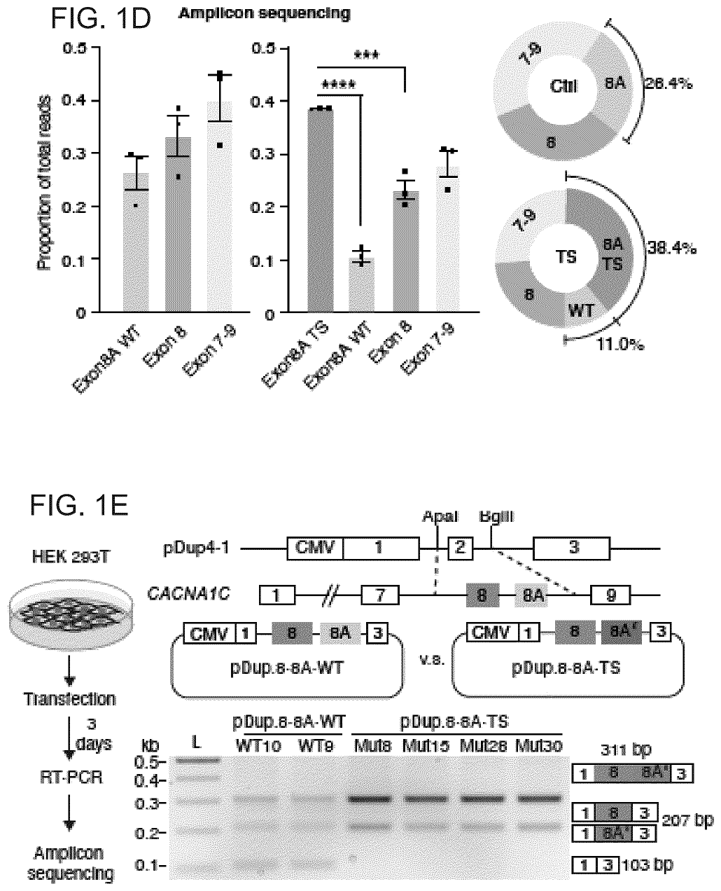

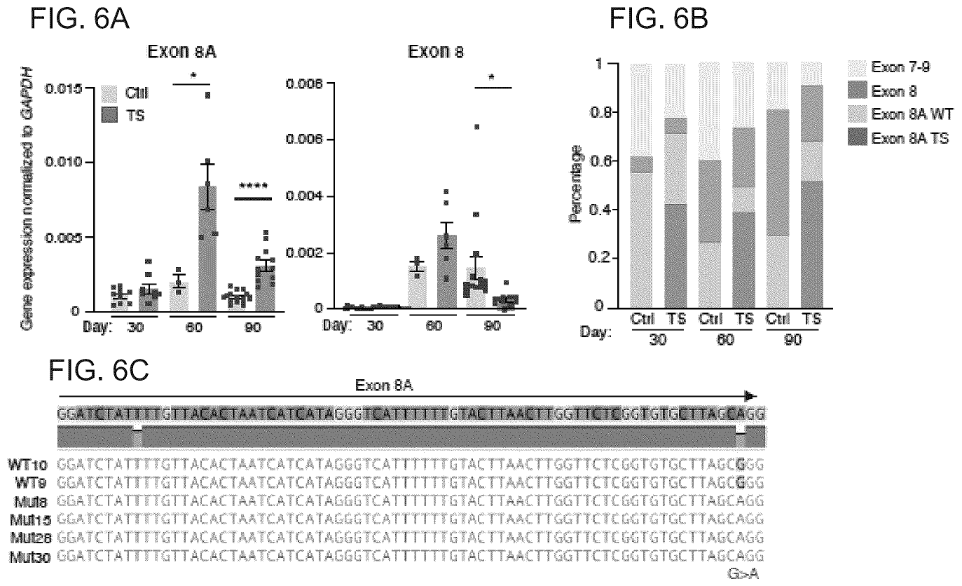

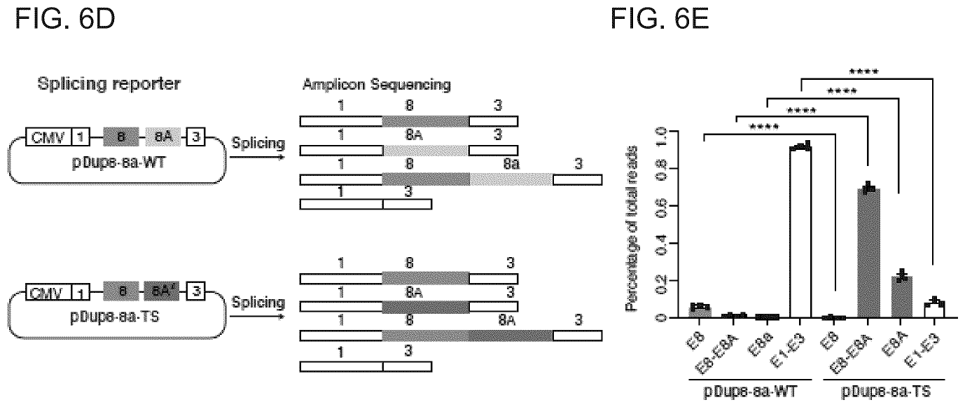

The TS mutation enhances splicing of the 8 A CACNA1C spliceform causing abnormal channel function in human cortical neurons. Exon 8 and exon 8 A are mutually exclusive, 104 nucleotide-long exons of the CACNA1C gene (FIGS. 1A-1B). During cortical differentiation in vitro, both in 2D cultures (2,5) and in 3D human cortical organoids (hCO) (FIG. 6A), 8 A is expressed at higher levels at early stages, and this changes in favor of 8 over time (FIG. 6A, P<0.001,). Interestingly, hCO derived from TS1 patients expressed considerably higher levels of exon 8 A compared to control hCO at day 60-90 of differentiation. A restriction fragment length polymorphism (RFLP) assay that uses the BamHI restriction enzyme selectively cut exon 8 further confirmed this finding (FIG. 1C) raising the possibility that the G406R mutation may interfere with splicing, which could amplify disease phenotypes.

To verify if the TS mutation affects the splicing machinery to cause increased levels of exon 8 A, the mRNA composition of exon 8 A in TS hCO was first analyzed, which contains both wildtype (WT) and p.G406R exon 8 A alleles. Equal amount of WT and p.G406R alleles of exon 8 A would suggest that the splicing machinery does not distinguish the TS allele from the WT allele. However, by sequencing the amplicons spanning from exon 7 to exon 9 from cDNA of both TS and WT hCO, it was discovered that the elevated exon 8 A expression in the TS samples preponderantly contains the G406R allele (FIG. 1D and FIG. 6B). It was then asked whether this TS-related enhanced splicing depends on the cellular or genomic context. Two minigene splicing reporters were generated in which ˜1 kb DNA fragments spanning exon 8 and 8 A (either WT or TS) were inserted into the pDup4-1 reporter backbone (FIG. 1E and FIGS. 6C-6D; pDup8-8 A^{WT} and pDup8-8 A^{TS}). Transfection and amplification of these two vectors in HEK293T cells revealed strikingly different splicing outcomes (FIGS. 6D-6E, P<0.0001). The pDup8-8 A^{WT} mostly transcribed exon 8, while the mutant pDup8-8 A^{TS} preferentially transcribed exon 8 A indicating that the TS mutation is sufficient to shift CACNA1C splicing in favor of exon 8 A. Together, these experiments demonstrate that the developmentally regulated TS 8 A CACNA1C variant directly enhances its incorporation by interfering with splicing.

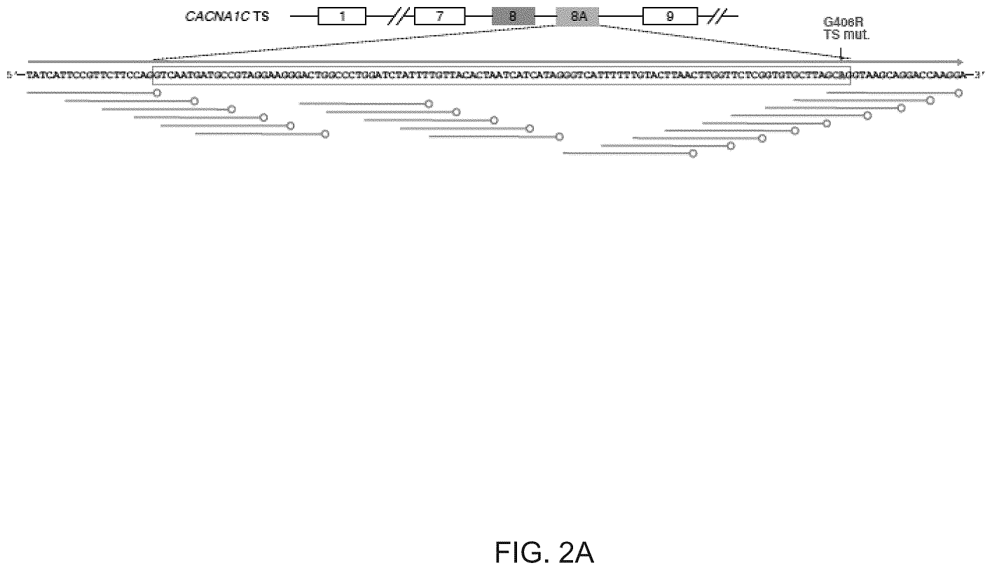

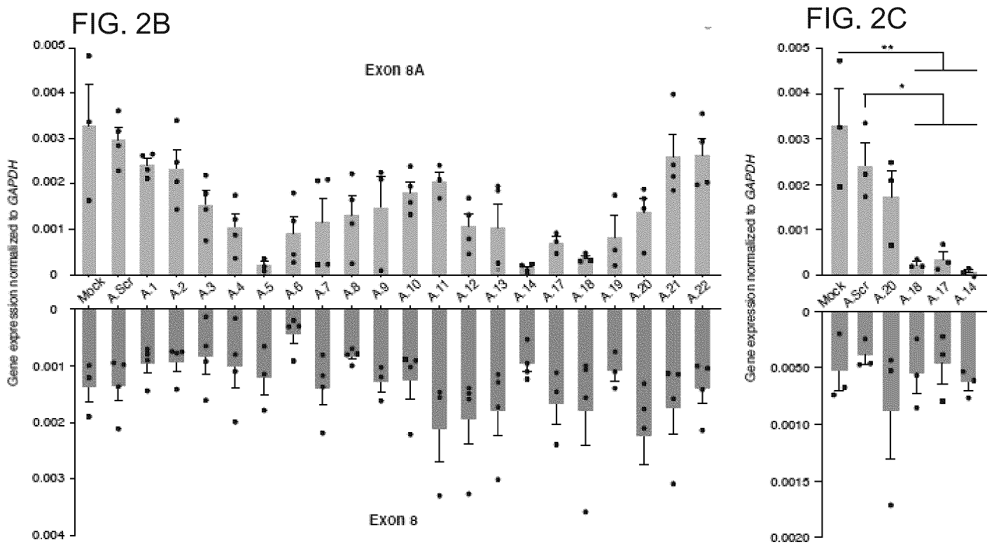

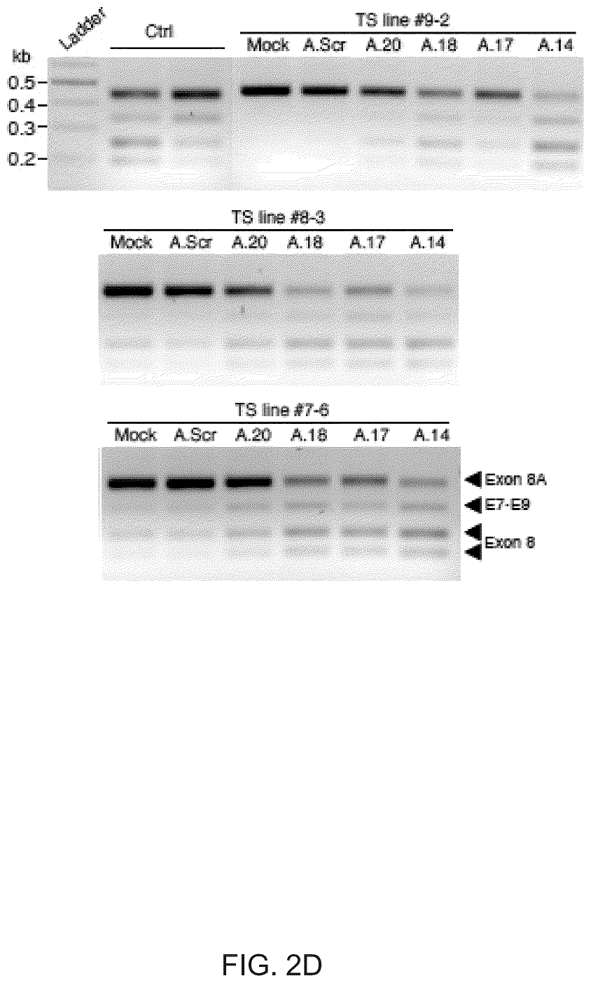

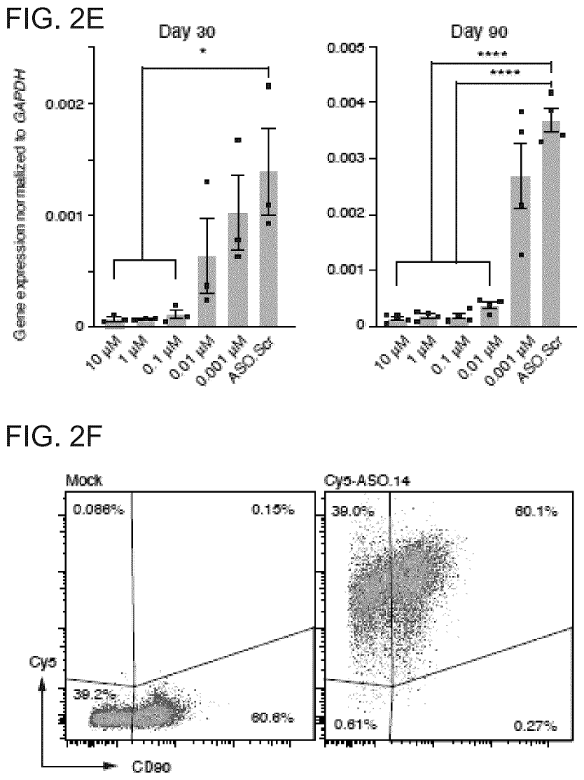

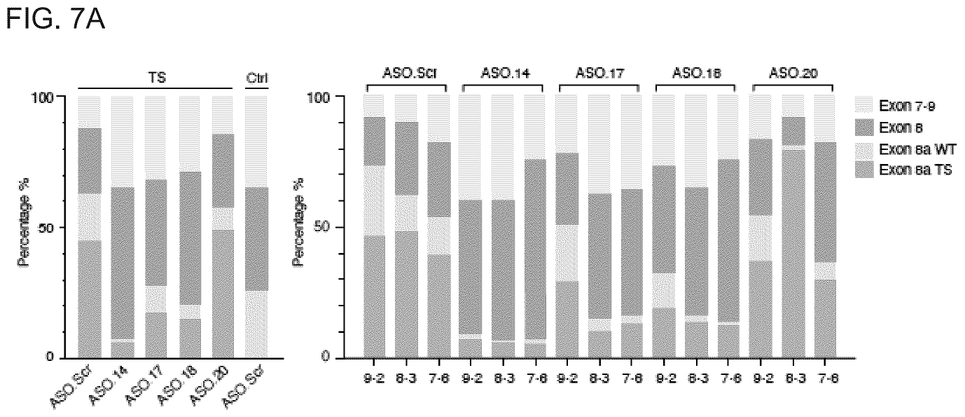

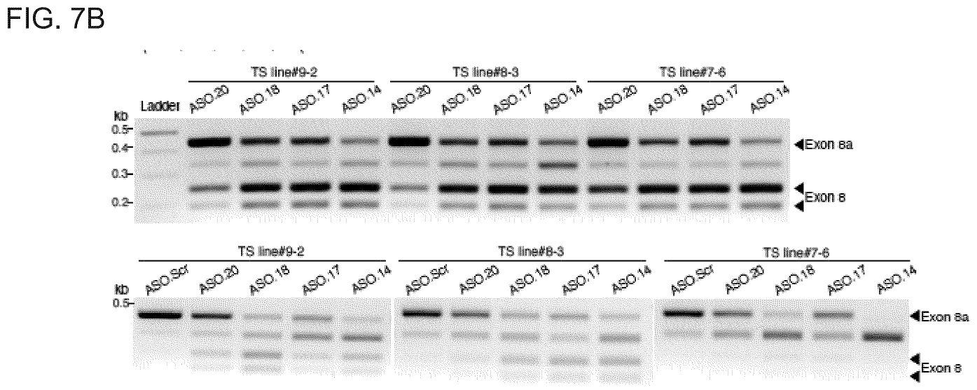

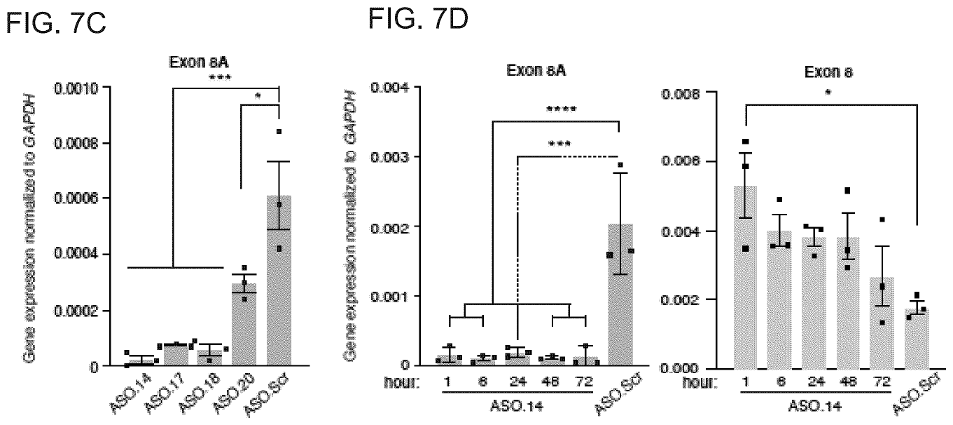

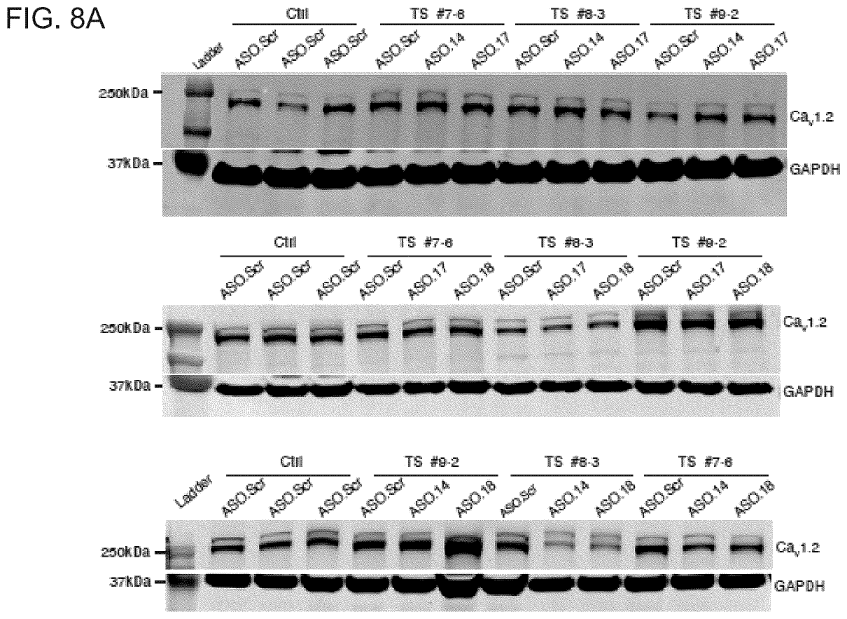

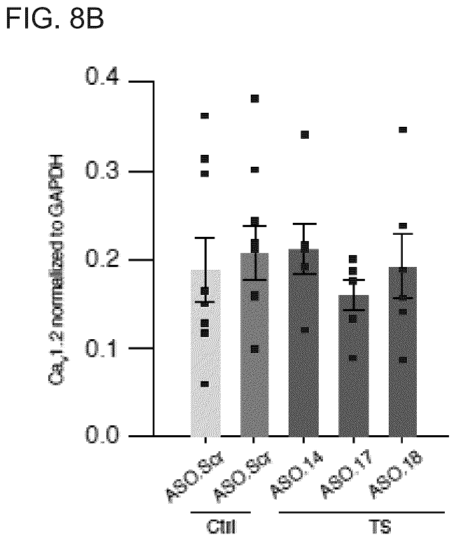

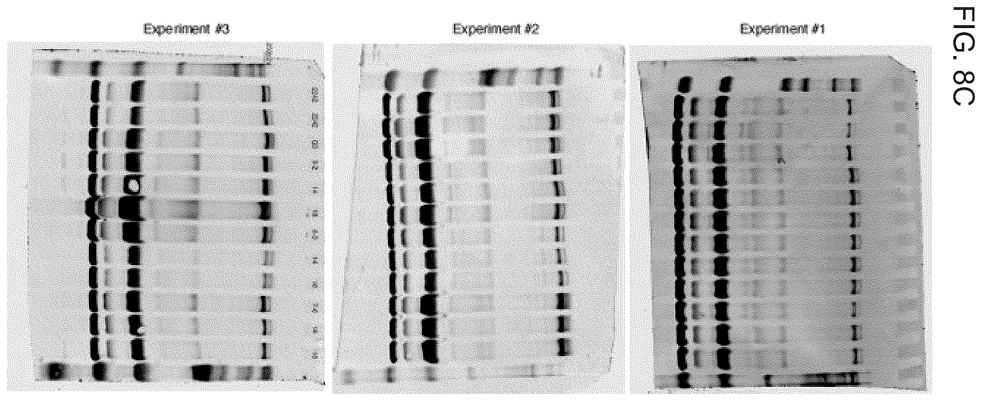

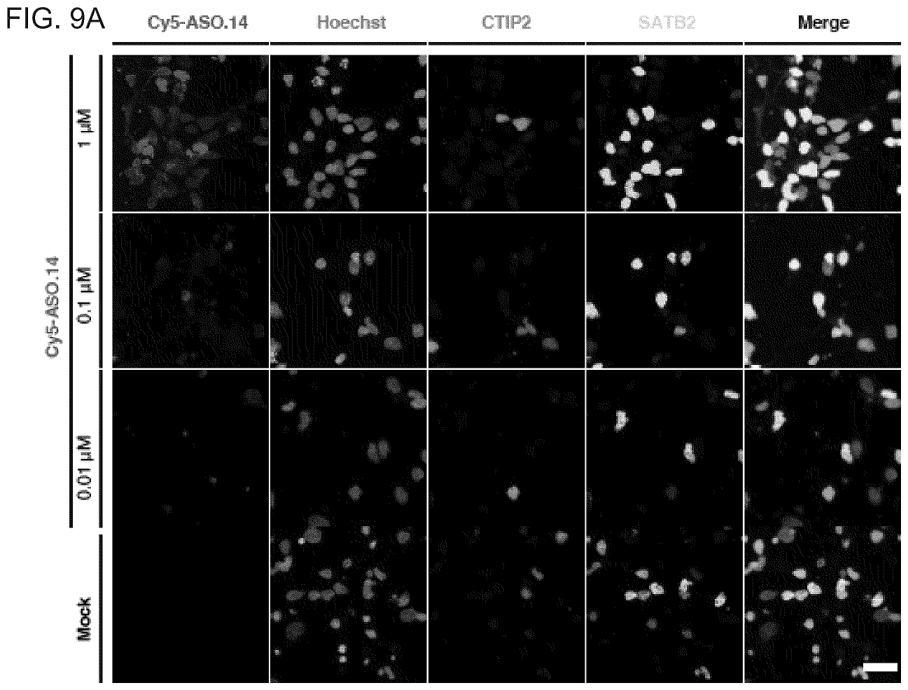

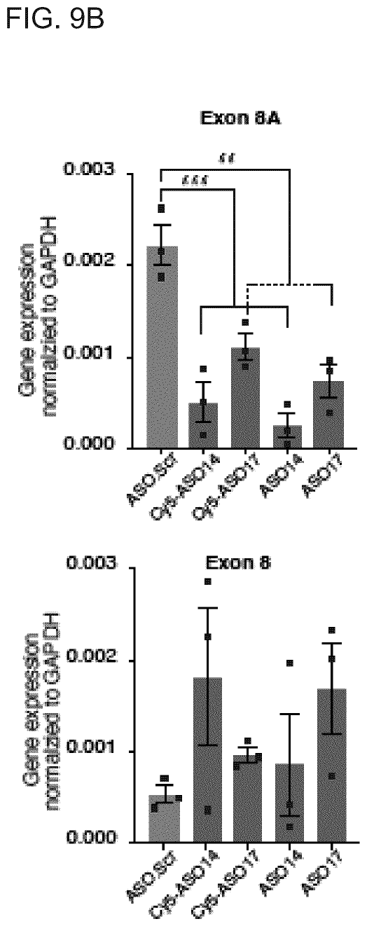

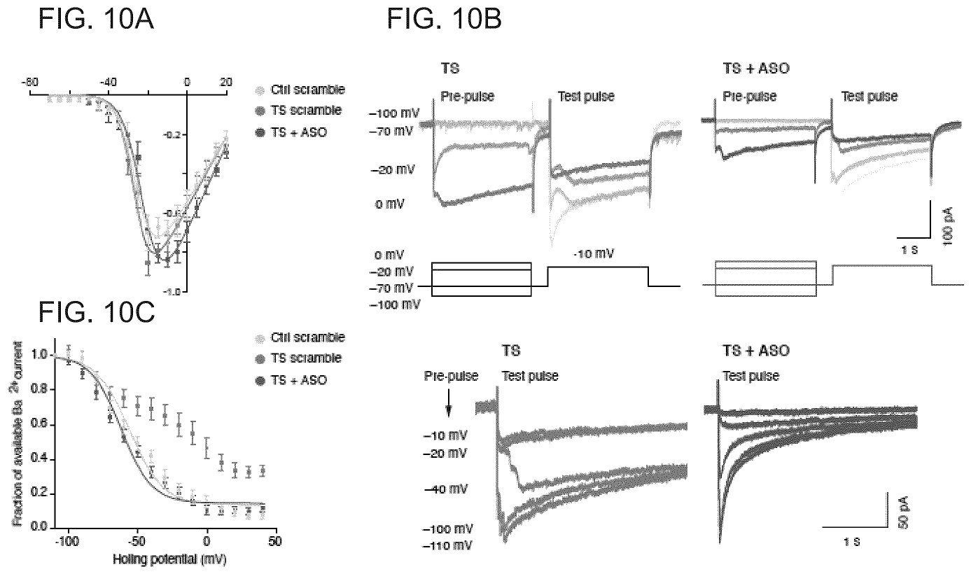

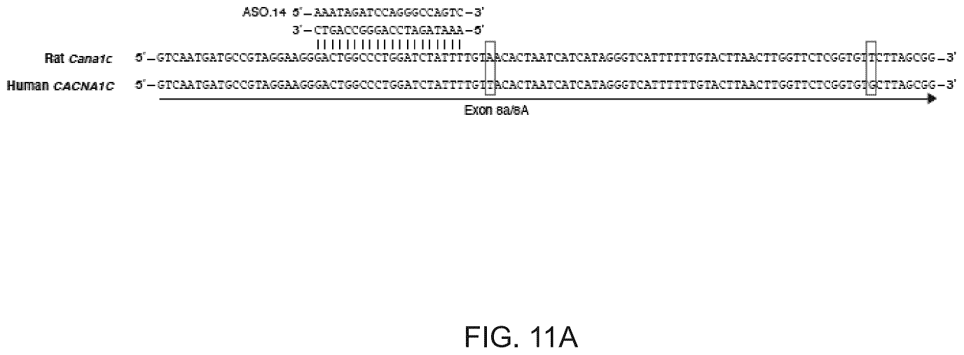

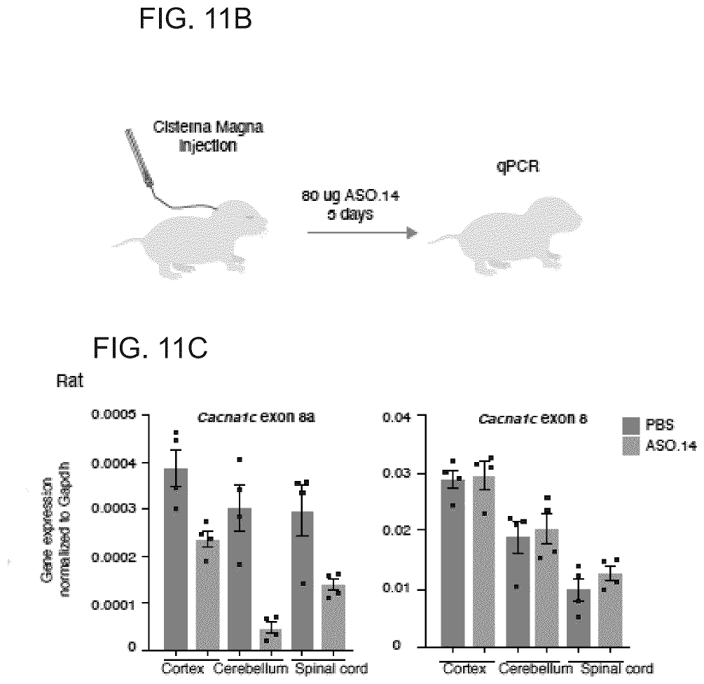

Screening identifies ASOs that can reduce exon 8 A in favor of exon 8 CACNA1C isoforms in human neurons. To screen ASOs that could modify exon 8 splicing, an ASO walking strategy was designed with 5 nt spacing, which covered exon 8 A (Table 1). ASOs with a universal 2′-O-methoxyethylribose (MOE) modification were used to avoid potential degradation of the CACNA1C mRNA (FIG. 2A). TS hiPS cells were differentiated into hCO, dissociated them into 2D neural cultures, and added 10 μM ASO targeting either exon 8 A or a scrambled control ASO. Three days later, RT-qPCR of exon 8 and exon 8 A showed that several ASOs induced robust down-regulation of exon 8 A without changing exon 8 expression (FIG. 2B). To validate these results in 3D hCO, the top four ASOs were selected (ASO.14, ASO.17, ASO.18 and ASO.20). Exposure for 3 days to ASOs in 3D cultures, yielded selective exon 8 A downregulation, by RT-qPCR analysis (FIG. 2C, P<0.001) and RFLP (FIG. 2D). Sequencing these amplicons further confirmed that ASO.14, ASO.17, and ASO.18 targeted and downregulated exon 8 A in hCO derived from 3 TS patients (FIG. 7A). These effects were long-lasting as one single ASO exposure effectively suppressed 8 A even at 15- and 30-days post-treatment (FIG. 7B, P<0.05, and c). Moreover, this 8 A to 8 switch was not associated with changes in the total amount of Caᵥ1.2 protein as indicated by western blots in hCO (FIGS. 8A-8C).

TABLE 1
ASOs used in this study

| | | | | SEQ |
|---|---|---|---|---|
| | Sequence | ASO | ASO | ID |
| Name | (5′-3′) | Chemistry | Length | NO: |
| ASO.CA1 | TCCTTGGTCC | Uniform MOE | 20 | 1 |
| | TGCTTACCTG | | | |
| ASO.CA2 | GGTCCTGCTT | Uniform MOE | 20 | 2 |
| | ACCTGCTAAG | | | |
| ASO.CA3 | TGCTTACCTG | Uniform MOE | 20 | 3 |
| | CTAAGCACAC | | | |
| ASO.CA4 | ACCTGCTAAG | Uniform MOE | 20 | 4 |
| | CACACCGAGA | | | |
| ASO.CA5 | CTAAGCACAC | Uniform MOE | 20 | 5 |
| | CGAGAACCAA | | | |
| ASO.CA6 | CACACCGAGA | Uniform MOE | 20 | 6 |
| | ACCAAGTTAA | | | |
| ASO.CA7 | CGAGAACCAA | Uniform MOE | 20 | 7 |
| | GTTAAGTACA | | | |
| ASO.CA8 | ACCAAGTTAA | Uniform MOE | 20 | 8 |
| | GTACAAAAAA | | | |
| ASO.CA9 | TGACCCTATG | Uniform MOE | 20 | 9 |
| | ATGATTAGTG | | | |
| ASO.CA10 | CTATGATGAT | Uniform MOE | 20 | 10 |
| | TAGTGTAACA | | | |
| ASO.CA11 | ATGATTAGTG | Uniform MOE | 20 | 11 |
| | TAACAAAATA | | | |
| ASO.CA12 | TAGTGTAACA | Uniform MOE | 20 | 12 |
| | AAATAGATCC | | | |
| ASO.CA13 | TAACAAAATA | Uniform MOE | 20 | 13 |
| | GATCCAGGGC | | | |
| ASO.CA14 | AAATAGATCC | Uniform MOE | 20 | 14 |
| | AGGGCCAGTC | | | |
| ASO.CA17 | CAGTCCCTTC | Uniform MOE | 20 | 15 |
| | CTACGGCATC | | | |
| ASO.CA18 | CCTTCCTACG | Uniform MOE | 20 | 16 |
| | GCATCATTGA | | | |
| ASO.CA19 | CTACGGCATC | Uniform MOE | 20 | 17 |
| | ATTGACCTGG | | | |
| ASO.CA20 | GCATCATTGA | Uniform MOE | 20 | 18 |
| | CCTGGAAGAA | | | |
| ASO.CA21 | ATTGACCTGG | Uniform MOE | 20 | 19 |
| | AAGAACGGAA | | | |
| ASO.CA22 | CCTGGAAGAA | Uniform MOE | 20 | 20 |
| | CGGAATGATA | | | |

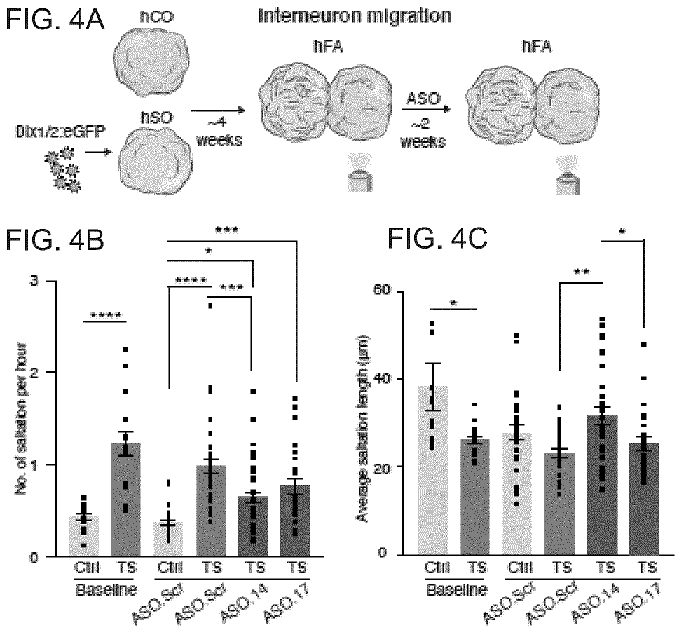

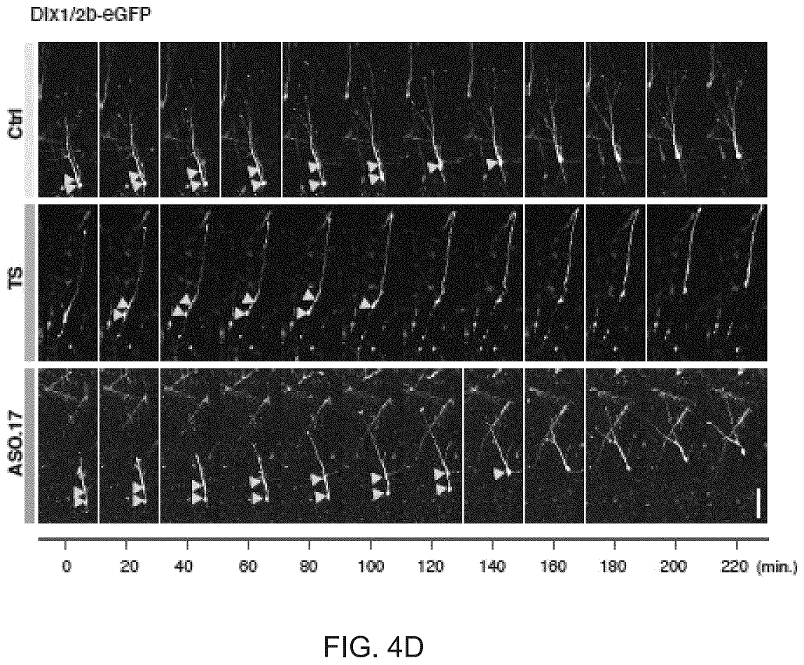

To study the pharmacodynamics of these ASOs, hCO were treated with different concentrations of ASO.14 at differentiation day 30 and day 90. A dose-dependent decrease of exon 8 A expression was observed (FIG. 2E, P<0.05 for day 30, P<0.00001, for day 90). hCO were then treated with ASO.14 and performed RT-qPCR analysis at 1, 6, 24, 48 and 72 hours after exposure. Surprisingly, it was found that ASO treatment affected splicing as early as 1-hour post-exposure in vitro (FIG. 2D, P<0.001). Finally, to prove that the ASO entered the cells, ASO.14 was labeled with Cy5 and quantified the Cy5-positive cells isolated from hCO by flow cytometry. Most cells, including CD90-expressing neurons, were Cy5⁺ (FIG. 2F). Moreover, treatment with Cy5-ASO correlated well with a dose-dependent Cy5 fluorescence by immunostaining (FIGS. 4A-4B). This indicates that several ASO can enter human neurons and effectively, in a dose-dependent and within a short period of time, reduced exon 8 A expression in Timothy syndrome.

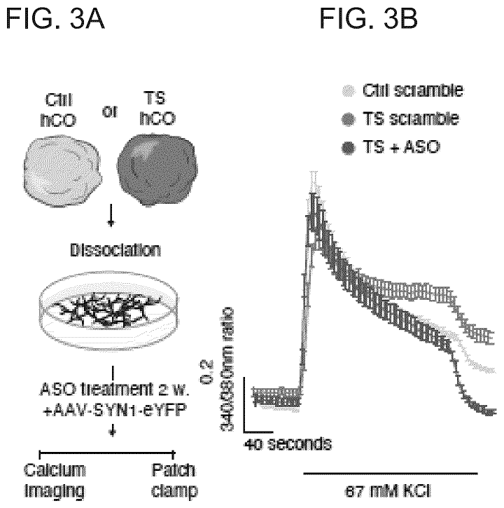

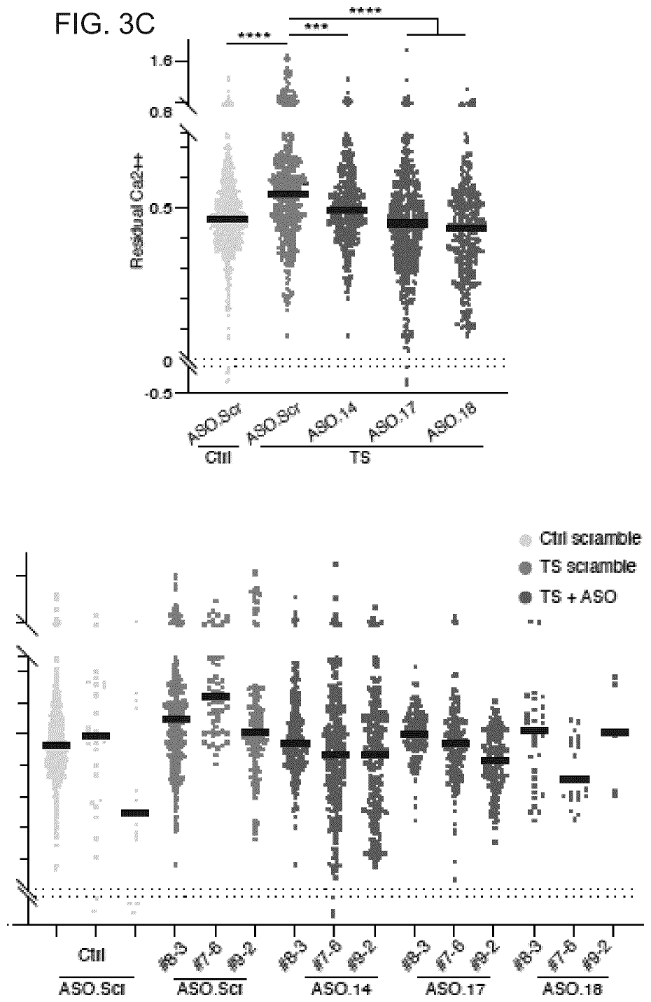

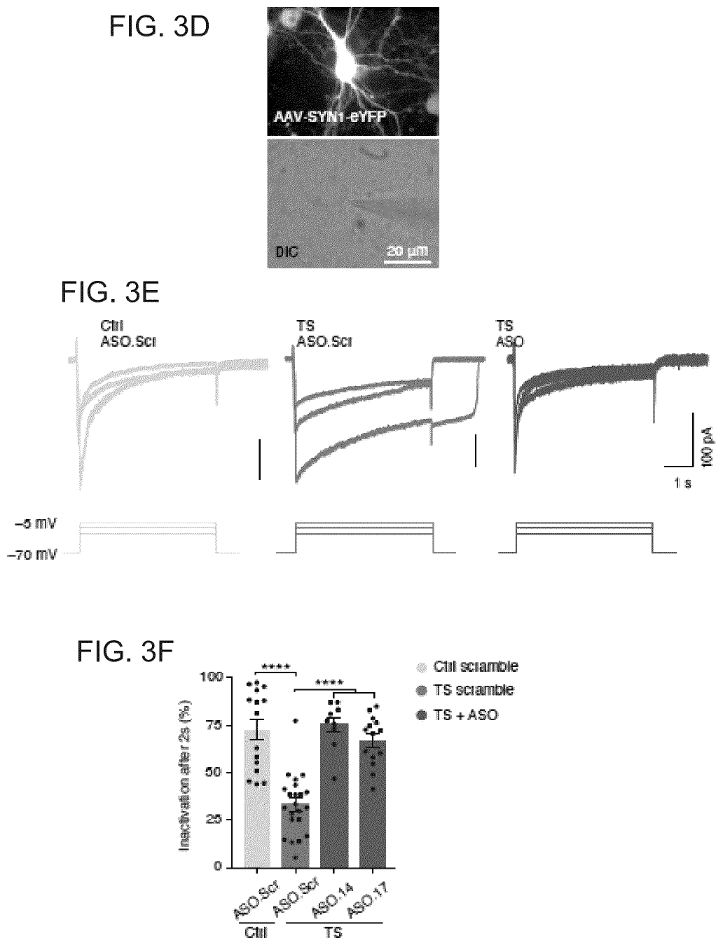

ASO rescued delayed inactivation and interneuron migration defects in TS hCO and hFAs. We previously demonstrated that TS cortical neurons display delayed inactivation of barium currents, increased intracellular calcium after depolarization, and impaired interneuron migration (3). To test whether altering exon 8 A/8 splicing via ASOs could restore Caᵥ1.2 channel function, TS hCO neurons were exposed to ASO.14, ASO.17, ASO.18 or ASO.Scr, and compared them to control hCO neurons exposed to ASO.Scr in a Fura-2 AM calcium imaging assay (FIG. 3A). As expected, TS neurons displayed increased residual calcium following depolarization as compared to control neurons (FIGS. 3B-3C, P<0.0001). All 3 selected ASOs restored residual calcium to a control level, suggesting that ASOs can functionally rescue of the Caᵥ1.2 channel. (FIGS. 3B-3C, P<0.001). ASO.14 and ASO.17 were then applied (which has similar effects to ASO.18) to TS and control hCO and performed whole-cell patch clamping of hSyn1-YFP neurons (FIGS. 3A-3D). TS neurons had delayed inactivation of barium currents as measured by the percent of channel inactivation after 2 seconds of current clamp (FIG. 3E). This defect was rescued by both ASO.14 and ASO.17 (FIG. 3F, P<0.0001).

It was previously discovered that TS interneurons migrate abnormally in hFA (3). To investigate whether ASOs can correct this cellular migratory defect in 3D cultures, TS and control hCO and hSO were derived and hFA were generated and labeled interneurons with a cell specific reporter (LV.Dl×1/2::eGFP), as previously shown (3) (FIG. 4A). Three to four weeks post-assembly, the saltation frequency was imaged and the average saltation length of TS and control interneurons at baseline were quantified; hFA were then exposed to ASO.14, ASO.17 or ASO.Scr and performed another imaging experiment 2 weeks later. At baseline prior to ASO treatment, accelerated saltation frequency were found (FIG. 4B, P<0.001) and shortened saltation length in TS interneurons compared to control interneurons (FIG. 4C, P<0.05). Treatment with ASO.14 and ASO.17 reduced saltation frequency of TS interneurons (FIG. 4B, P<0.05) and increased saltation length (FIG. 4C, P<0.05). In summary, it was found that exposure to 8/8 A switching ASO effectively rescues channel function, calcium dynamics and cellular phenotypes in TS patient-derived cultures in vitro.

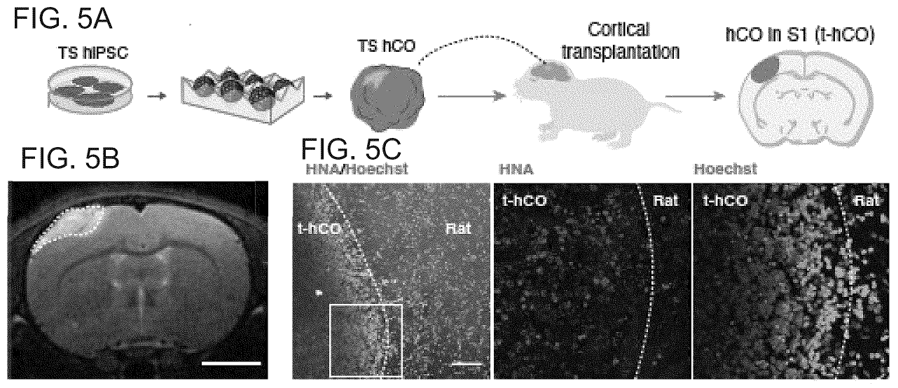

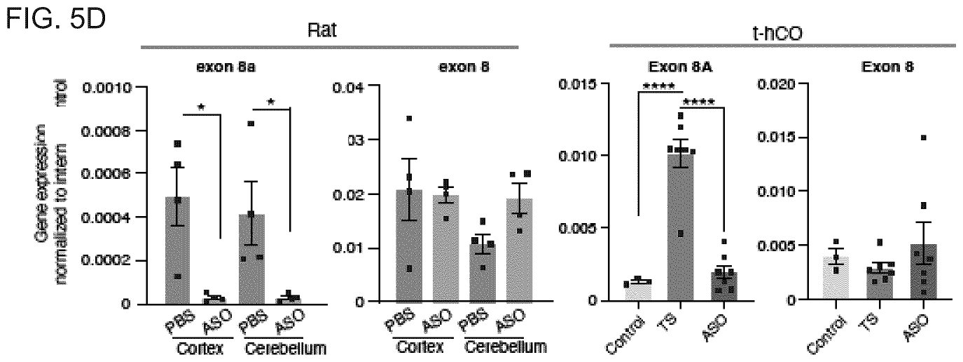

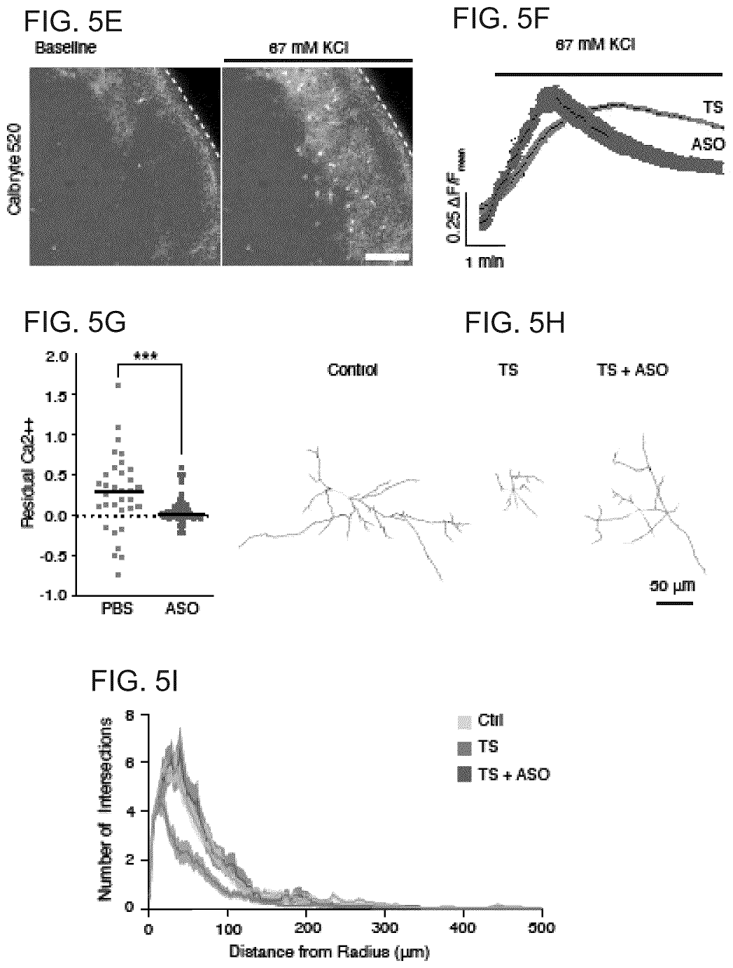

ASO delivery in vivo rescues TS related phenotypes in human cells. Encouraged by these findings and motivated to verify the translational potential of these ASOs in TS, their effect in an in vivo setting was verified. A transplantation strategy was recently developed into the developing cerebral cortex of early-postnatal rats that allows hCO to develop mature cell types integrate both anatomically and functionally into the rodent brain. This in vivo platform was applied to test delivery of ASO in vivo and their ability to rescue genetic and functional defects in cells from patients with TS1 (FIGS. 5A-5B).

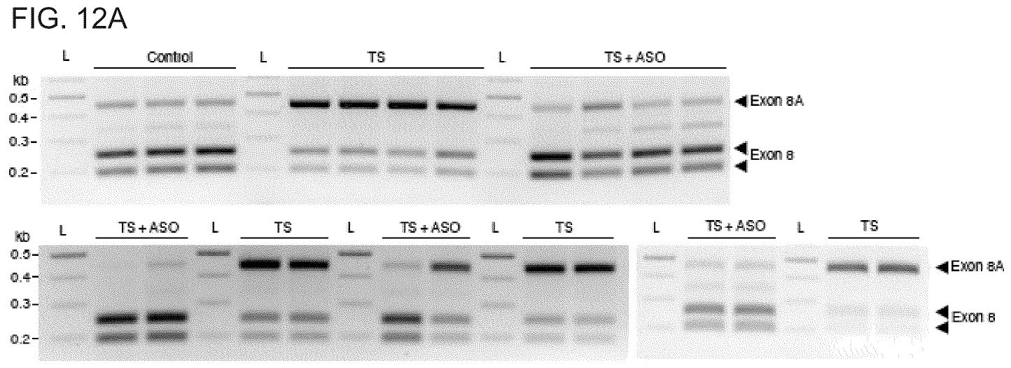

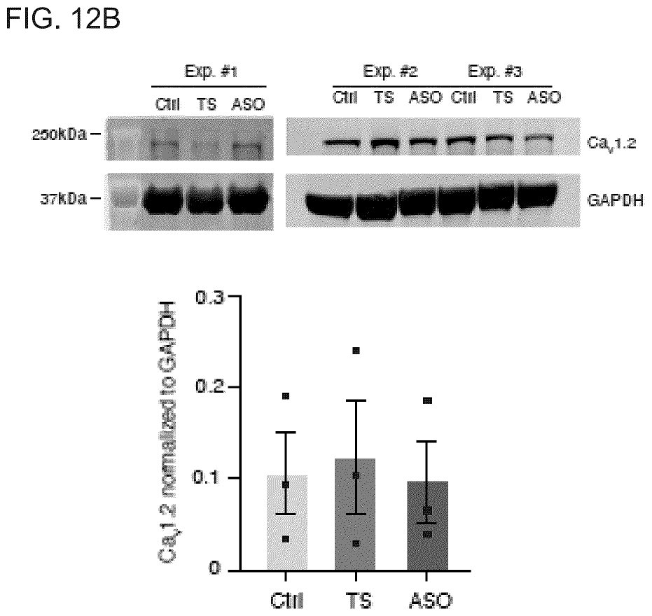

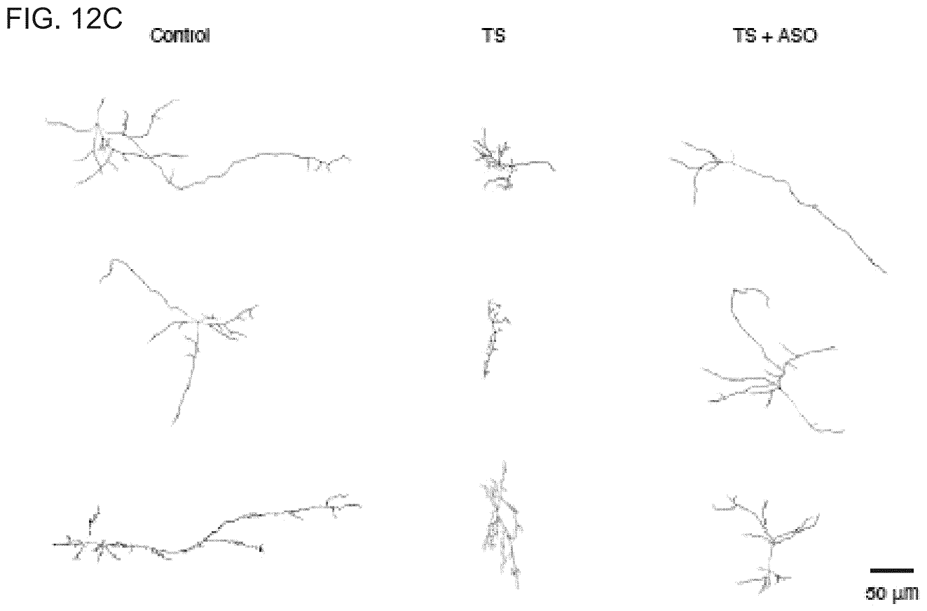

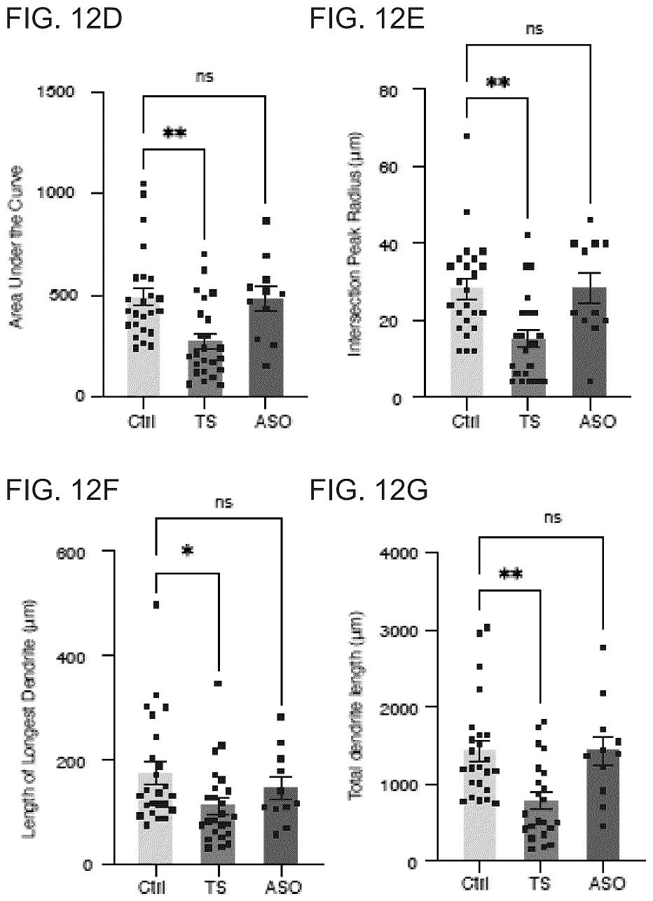

We next transplanted hCO from two individuals with TS and monitored t-CO and graft position by MRI and immunostaining (FIGS. 5B-5C). We then injected 300 μg ASO.14 into the cisterna magna (FIG. 5D). Seven to nine days later, we extracted the hCO graft and found that CACNA1C exon 8 A in TS t-hCO had reduced expression (FIGS. 5D-5F, P<0.001, FIG. 12A). This was accompanied by a reduction in the Cacna1c exon 8 A expression in the rat cortex and cerebellum (FIG. 5E, P<0.05). Similar to in vitro ASO experiments, overall CaV1.2 levels were not affected (FIG. 12B). This experiment indicates that ASOs can be delivered intracranially and can effectively modulate splicing in the human transplanted cells. Lastly, we attempted to verify the effects of ASO administration on cellular dysfunction resulting from the TS1 mutation. To do this, we extracted t-hCO, sliced the tissue and performed ex vivo calcium imaging using the calcium indicator Calbryte 520 AM (FIG. 5H). As expected, we found that TS neurons had increased post-depolarization residual calcium compared with WT t-hCO neurons, but that ASO.14 significantly reduced residual calcium, albeit not completely back to WT baseline (FIGS. 5I-5J). Finally, TS is associated with activity-dependent dendrite morphology defects and this can be detected in patient-derived cortical neurons following transplantation in vivo. To test whether ASOs can rescue this morphological phenotype, we traced neurons using Golgi staining in t-hCO at 14 days post-ASO injection. We found that ASO.14 corrected the TS neuron morphology in vivo (FIGS. 12C-12G).

These experiments indicate that ASOs induce splicing modulation of human CACNA1C both in vitro and in vivo and thereby rescue molecular and cellular phenotypes of TS1.

### DISCUSSION

Developing therapies for neuropsychiatric disorders has been a significant challenge due to the inaccessibility of human brain tissue. Despite an understanding of the genetic cause and of some of the molecular mechanisms of TS, there still is not a promising therapeutic avenue. L-type calcium channel blockers do not restore many of the cellular phenotypes in TS and roscovitine has extensive off target effects (2,11). Some, but not all, of the defects identified with human cellular models have been recapitulated in a mouse (16) expressing the channel with the Timothy syndrome mutation (albeit the mutation is in exon 8), suggesting that species-specific differences in gene regulation can change the cellular phenotypes associated with a disease.

Here, a potential therapeutic strategy was developed for a severe neurodevelopmental caused by a point mutation in an alternatively spliced exon. To do this, splicing profiles in human neurons were first investigated and it was found the persistent exon 8 A in TS is biased towards the TS variant, which likely amplifies defects downstream of this dysfunctional calcium channel. Subsequently ASOs were screened and identified that can effectively modulate splicing in TS to reduce 8 A in favor of 8 without changing the level of Caᵥ1.2 protein. It was demonstrated that these ASOs can in a dose- and time-dependent manner rescue ion flux kinetics, calcium dynamic and cellular movement defects in human neurons derived from 3 TS1 patients in human organoid and assembloid models. Lastly, it was shown that ASOs can be delivered in vivo using a novel transplantation platform that was developed and, importantly, can rescue the splicing and calcium defects in human neurons that have been integrated into the rat cerebral cortex.

Nonetheless, the proof-of-concept study, which includes a combination of in vitro and in vivo studies with human patient-derived 3D multi-cellular models, illustrates how this platform could be used for studying other neuropsychiatric diseases, and for evaluating the efficiencies and safeties of therapies, including but not limited to ASOs, viral vectors and small molecules. This will be particularly relevant when animal models are not available or do not fully recapitulate human pathology.

Methods

Culture of hiPS cells and HEK cells. The hiPS cells in this study were described and previously validated (2,3). Three hiPS cell lines were derived from fibroblasts collected from 3 healthy individuals and 3 individuals with TS. Approval for this study was obtained from the Stanford IRB panel, and informed consent was obtained from all subjects. The hiPS cells were cultured in the feeder-free Essential 8 Medium (E8; ThermoFisher Scientific; Cat. No.: A1517001) without antibiotics and kept in wells of six-well plates (Corning; Cat. No.: 3506) coated for 1 hour at room temperature with Vitronectin Recombinant Human Protein (VTN-N; ThermoFisher Scientific; Cat. No.: A14700) diluted 1:100 to a final concentration of 5 ng ml⁻¹ in DPBS, no calcium, no magnesium (ThermoFisher Scientific; Cat. No.: 14190136). To facilitate the passaging, the hiPS cells were first washed with DPBS and then incubated with 0.5 mM ethylenediaminetetraacetic acid (EDTA; Invitrogen; Cat. No.: 15575020) in DPBS at room temperature for 7 minutes. After the removal of EDTA solution, cells were seeded in new wells of 6-well plates coated with VTN-N and containing E8 medium. The hiPS cells used in this study were maintained free of Mycoplasma and were kept at 37° C. in a humidified-air atmosphere with 5% CO2. HEK cells were cultured in Dulbecco's Modified Eagle Medium (DMEM; Gibco; Cat. No.: 10313021) supplemented with 10% Fetal Bovine Serum (FBS; Corning; Cat. No.: 35016CV) and 1× GlutaMAX (ThermoFisher Scientific; Cat. No.: 35050061).

Generation of hCO and hSO from hiPS cells. The generation of hCO, hSO, and hFA was performed as previously described (3, 17, 18). In brief, hiPS cells were incubated with Accutase (Innovate Cell Technologies, AT-104) at 37° C. for 7-8 minutes and dissociated into single hiPS cells. The single cell suspensions were collected in a 50-ml Falcon tube and the cell pellets were obtained via centrifuge for 300 g for 3 minutes. Cell numbers were counted after resuspending the cell pellets. Approximately 3×10⁶ cells in 2 ml E8 media supplemented with ROCK inhibitor Y-27632 (10 μM; Selleckchem; Cat. No.: S1049) were added per well of the AggreWell 800 plate (STEMCELL Technologies; Cat. No.: 34815). The plates were then centrifuged at 100 g for 3 min to allow the cells to sink into the bottoms of the wells (day 0). 24 h after the cell aggregation (day 1), spheroids were dislodged by pipetting (with a cut end of a P1000 tip) and transferred into the ultra-low-attachment plastic dishes (Corning, 3262) in Essential 6 medium (E6; Life Technologies; A1516401) supplemented with dorsomorphin (DM; 2.5 μM; Sigma-Aldrich; Cat. No.: P5499) and SB-431542 (SB; 10 μM; Tocris; Cat. No.:1614). From day 2 to day 5, E6 medium was changed daily and was supplemented with DM and SB. Additionally, the Wnt pathway inhibitor XAV-939 (XAV; 1.25 μM; Tocris; Cat. No.: 3748) was added together with DM and SB. On the sixth day in suspension, the basal media was switched to neural media consisting of Neurobasal A (Life Technologies; Cat. No.: 10888), B-27 supplement without vitamin A (B27; Life Technologies, Cat. No.: 12587), GlutaMax (1:100, Life Technologies; Cat. No.: 35050) and 10 U/ml Penicillin-Streptomycin (Gibco; Cat. No.: 15140122). From day 6 to day 24, the neural medium was supplemented with 20 ng ml-1 epidermal growth factor (EGF; R&D Systems, Cat. No.: 236-EG) and 20 ng ml⁻¹ basic fibroblast growth factor (FGF; R&D Systems; Cat. No.: 233-FB) for 19 d (until day 24), with medium changed daily from day 6 to day 17 and every other day till day 24. From day 25 to day 42, the neural medium contained 20 ng ml⁻¹ brain-derived neurotrophic factor (BDNF; Peprotech; Cat. No.: 450-02) and 20 ng ml⁻¹ NT3 (Peprotech; Cat. No.:450-03), with medium changes every other day. From day 43 on, the hCSs were cultured with only neural medium without growth factors. The generation of hSSs differs with that of hCSs in that, from day 6 to day 12, the neural medium was supplemented with XAV (1.25 μM) besides EGF and FGF; from day 13 to day 24, the neural medium was supplemented with XAV (1.25 μM) and SAG (100 nM; EMD Millipore; Cat. No.: 566660), besides EGF and FGF.

Antisense oligonucleotides. ASO were 20 nt-long in a phosphorothioate (PS) backbone and with a uniform 2′-O-methoxy-ethyl (MOE) modification. 5-Methylcytosine was used during synthesis instead of cytosine. ASOs tested on the hiPS cell-derived forebrain organoids were purified by standard desalting followed by Na⁺ salt exchange. These ASOs were reconstituted in nuclease-free water at a concentration of 1 mM and stored at −20° C. thereafter. All ASOs used in this study were manufactured by Integrated DNA Technologies (IDT). Cy5-labeled ASOs were synthesized by adding the Cy5 to the 5′ of the ASO (IDT), followed by HPLC purification and Na⁺ salt exchange. ASOs used are listed in Table 1.

Recombinant DNA and viruses. pDup4-1 is from Addgene (plasmid #23022) was used as the backbone for the minigene splicing reporter. pDup4-1 was digested with Apal and Bglll (New England Biolabs) and the resulting 4595-bp fragment was purified after being loaded on a 1% agarose gel using the QIAquick PCR Purification Kit (Qiagen; Cat. No.: 28106). The genomic DNA of the TS hiPS cells was purified with a DNeasy Blood & Tissue Kit (Qiagen; Cat. No.: 69506). Amplicons encompassing the exon 8 and the exon 8 A of CACNA1C were amplified with the GoTaq Long PCR Master Mix (Promega; Cat. No.: M4021). Purified PCR products were digested with Apal and Bglll. After another round of purification, the DNA was dephosphorylated with FastAP Thermosensitive Alkaline Phosphatase (Thermofisher Scientific; Cat. No.: EF0654), then ligated to the pDup4-1 backbone mentioned above using the T4 DNA ligase (Thermofisher Scientific; Cat. No.: EL0011). After transformation (One Shot Stbl3 Chemically Competent E. coli, Thermofisher Scientific; Cat. No.: C737303), colonies were picked for sequence verification.

RNA extraction and qPCR. For all samples, RNA was extracted by RNeasy Plus Mini Kit (Qiagen; Cat. No.: 74136). Unless otherwise mentioned, reverse transcription was done using the SuperScript Ill First-Strand Synthesis SuperMix for qRT-PCR (Invitrogen; Cat. No.: 11752050) according to the manufacturer's instructions. qPCR was performed on QuantStudio 6 Flex Real-Time PCR system (ThermoFisher Scientific; Cat. No.: 4485689) using the SYBR Green PCR Master Mix (ThermoFisher Scientific; Cat. No.: 4312704).

Transcript analysis of the CACNA1C exon 8 and exon 8 A. Restriction fragment length polymorphism (RFLP) analysis of CACNA1C exon 8 and exon 8 A was performed on PCR fragments amplified from cDNA. The DNA was purified using the AMPure XP beads (Beckman Coulter; Cat. No. A63881) according to manufacturer's instructions. The purified DNA was digested with BamHI (ThermoFisher Scientific; Cat. No: ER0055) at 37° C. for 3 hours and loaded on 2% agarose gel. Gel images were taken on Gel Doc XR+ imager (Bio-Rad; Cat. No. 1708195). For next generation sequencing (NGS) analysis of the transcripts, primers with the illumine adapter were used to amplify the region encompassing the exon 7 to exon 9. After beads purification, the DNA was eluted in water and sent for sequencing using the Genewiz Amplicon-EZ module. The NGS analysis of the minigene splicing reporter were performed similarly by amplifying the minigene transcripts on the cDNA of transfected HEK cells at 3 days post transfection.

Transfection of HEK cells. Approximately 30,000 to 75,000 HEK cells were seeded per well of 24-well plates (Corning; Cat. No.:). Next day, plasmids were mixed with 1 mg ml⁻¹ of PEI MAX (Polysciences; Cat. No.: 24765-1) in 50 μl of a 150 mM NaCl solution. After ˜10 s vigorous vortexing, the plasmid mixtures were incubated for 15 minutes at RT and were added to the wells thereafter.

Dissociation for monolayer culture. Dissociation of hCO for monolayer culture was performed as previously described with minor optimizations (4). Coverslips were coated with approximately 0.001875% polyethylenimine (PEI; Sigma-Aldrich; Cat. No.: 03880) for 1 hour at 37° C., washed 4 times with water and dried. On the day of dissociation, 4-6 hCO per hiPS cell line were transferred in a well of 6-well plates (Corning; Cat. No.: 3506) and incubated for 45 minutes at 37° C. with 3 ml enzymatic dissociation solution consisting of 30 U/ml papain (Worthington Biochemical; Cat. No.: LS003127), 1× EBSS (Millipore Sigma; Cat. No.: E7150), 0.46% D(+)-Glucose, 0.5 mM EDTA, 26 mM NaHCO₃, 10 μM Y-27632, 125 U/ml Deoxyribonuclease I (Worthington Biochemical; Cat. No.: LS002007) and 6.1 mM L-cysteine (Millipore Sigma; Cat. No.: C7880). At the end of papain incubation, samples were collected to a 15-ml Falcon tube and centrifuged at 1,200 rpm for 1 minute. After removing the supernatant, the samples were washed with 1 ml inhibitor solution with 2% trypsin inhibitor (Worthington Biochemical; Cat. No.: LS00308) and resuspend in 1 ml of the same solution for trituration. At the end of trituration, 1 ml inhibitor solution with 4% trypsin inhibitor was slowly added beneath the cell suspension to create a gradient layer. The gradient solution was centrifuged at 1,200 rpm for 5 minutes. Cell pellets were resuspended in culture media consisting of Neurobasal A supplemented with B27 and 10 μM Y-27632. Undissociated tissue was removed by passing the cell suspension through a 40 μm cell strainer (Corning; Cat. No.: 352340). Finally, dissociated cells were seeded on the coverslip at a density of 50,000 cells/coverslip in 1 ml culture media. The inhibitor solution differs with the enzyme solution in that it does not contain papain and EDTA. All centrifugation steps were performed at room temperature.

Calcium imaging. Fura-2 calcium imaging on monolayer hCO cells was performed as previously described (19-20). Briefly, cells were loaded with 1 mM Fura-2 acetoxymethyl ester (Invitrogen, F1221) for 30 minutes at 37 C in NM medium, washed with NM medium for 5 minutes and then transferred to a perfusion chamber (RC-20, Warner instruments) in low-potassium Tyrode's solution (5 mM KCl, 129 mM NaCl, 2 mM CaCl2, 1 mM MgCl2, 30 mM glucose, 25 mM HEPES, pH 7.4) on the stage of an inverted fluorescence microscope (Eclipse TE2000U; Nikon). After 0.5 minute of baseline imaging, low-potassium Tyrode's solution was perfused for 1 minute. Imaging was performed at room temperature (25 C) on an epifluorescence microscope equipped with an excitation filter wheel and an automated stage. Openlab software (PerkinElmer) and IGOR Pro (version 5.1, WaveMetrics) were used to collect and quantify time-lapse excitation 340 nm/380 nm ratio images at an imaging rate of ˜1 Hz, as previously described (19). Residual calcium was calculated as (C-A)/(B-A) where A is the baseline value (5th frame), B is the peak value following depolarization (manually determined) and C is the decay value (B+25th frame).

Patch-clamp recordings. Patch-clamp recordings were performed on cortical neurons dissociated from hCO, as previously described. hCO were dissociated at day 100-150. A few days after dissociation, cells were infected with AAV-DJ-SYN1-eYFP and 1 μM ASO was added a week after dissociation. Recordings were typically made 3-4 weeks after dissociation. Cells were identified as eYFP⁺ with an upright slice scope microscope (Scientifica) equipped with Infinity2 CCD camera and Infinity Capture software (Teledyne Lumenera). Recordings were performed with borosilicate glass electrodes with a resistance of 7-10 MΩ. For barium current recordings, the external solution contained 100 mM NaCl, 3 mM KCl, 2 mM MgCl₂, 20 mM BaCl₂, 25 mM TEA-CI, 4 mM 4-AP, 10 mM HEPES, 20 mM glucose, pH 7.4 with NaOH, 300 mOsm. Internal solution contained 110 mM CsMethylSO₃, 30 mM TEA-CI, 10 mM EGTA, 4 mM MgATP, 0.3 mM Na₂GTP, 10 mM HEPES, 5 mM QX314-CI, pH 7.2 with CsOH, 290 mOsm. Data were acquired with a MultiClamp 700B Amplifier (Molecular Devices) and a Digidata 1550B Digitizer (Molecular Devices), low-pass filtered at 2 kHz, digitized at 20 kHz and analyzed with pCLAMP (version 10.6, Molecular Devices). Cells were given a −10 mV hyperpolarization (100 ms) every 10 seconds to monitor input resistance and access resistance. Cells were excluded for analysis if they had a change >30%. The liquid junction potential was not corrected in the study.

For barium current recordings, cells were recorded in the presence of TTX (0.5 μM) to block sodium currents and were held at −70 mV in voltage-clamp and depolarizing voltage steps (5 seconds, from −70 mV to +20 mV) were given with an increment of 5 mV. Inactivation of barium current was calculated at 2 seconds in the maximal current (−20 mV to 0 mV for most cells). Leak subtraction was used to minimize the artifact of membrane resistance in MultiClamp 700B. I-V curves were fitted in Origin (OriginPro 2021b, OriginLab) with a Boltzmann exponential function:

I=Gₘₐₓ*(V−E_{Ba})/(18) where Gₘₐₓ is the maximal conductance of the calcium channels, E_{Ba} is the reversal potential of barium estimated by the curve-fitting program, V_{0.5} is the potential for half-maximal steady-state activation of the barium current, and K is a voltage-dependent slope factor.

For voltage-dependent barium current inactivation, cells were held at −70 mV. A series of pre-pulse voltage steps (3 seconds), from −110 mV to +40 mV with an increment of 10 mV were given. A test of voltage step (−10 mV or 0 mV, where the maximal current was recorded) was then given for another 3 seconds. Barium current inactivation was calculated as the relative current normalized to the current amplitude from the first test pulse. Voltage-dependent inactivation curves were fitted with exponential functions in Origin.

Interneuron migration and imaging analysis. At 45-50 days of differentiation, hSO were incubated with lentiviral particles LV.Dl×1/2b::eGFP in an Eppendorf tube overnight and then transferred to the wells of 24-well plate. After 3-7 days, hSO were incubated with an hCO in an Eppendorf tube supplemented with 1 ml media for generate hFA. The hFA were then cultured in a single well of the ultra-low attachment 24-well plate (Corning). Baseline imaging of interneuron migration was taken around 3-4 weeks after the formation of hFA. 1 μM ASOs was added to the hFA thereafter and re-imaged after another 2 weeks. All imaging were taken inside the confocal chamber at 37° C. in a humidified-air atmosphere with 5% CO2.

Immunostaining. Dissociated cells from TS hCO at 100-120 days of differentiation were plated on coverslips that were previously coated and placed in a well of 12-well plate. Different concentration of Cy5-ASO.14 were added into the well. After 3 days, the coverslips were first fixed for 10 minutes at room temperature with a solution containing 1 volume of culture media and 1 volume of fixation buffer containing 4% paraformaldehyde (PFA) and 4% sucrose in DPBS. Next, 2 volume of fixation buffer was added for an extra 20 minutes to finish the fixation step. After two rounds of washing with DPBS, coverslips were incubated for one hour with blocking buffer consisting of 0.3% Triton-X100, 10% normal donkey serum prepared with PBS. After removing the blocking buffer, primary antibody was added for overnight incubation at 4° C. Ctip2 antibody (Abcam, Cat. No. ab18465) and SATB2 antibody (Abcam, Cat. No. ab51502) were diluted in the blocking buffer at 1:300. Coverslips were washed twice with DPBS, then incubated with secondary antibody (1:1000 in blocking buffer, Donkey anti-rat Alexa 488, Thermofisher Scientific; Cat. No. A-21208, Donkey anti-mouse Alexa 568, Thermofisher Scientific; Cat. No. A10027) at room temperature for an hour. After another two rounds of wash with DPBS, Hoechst 33258 (Thermofisher Scientific, Cat. No. H3569) was added on the coverslip for 10 minutes, then rinsed by a final round of wash with DPBS. Finally, the coverslips were mounted on the slides (Fisherbrand Superfrost Plus Microscope Slides, Fisher Scientific; Cat. No. 12-550-15) using Aqua-Poly/Mount (Polysciences; Cat. No. 18606). Images were acquired on a confocal SP8 (Leica Microsystems) using a 20× objective.

Flow Cytometry. TS hCO were incubated with 1 μM Cy5.ASO.14 in wells of 24-well ultra-low attachment plate (Corning; Cat. No. 3473) for a total period of 2 days. The hCO were then dissociated and resuspended in 200 μl staining buffer containing 3% BSA and 0.5 mM EDTA. Cells were incubated with PE Mouse Anti-Human CD90 (BD Biosciences; Cat. No. 555596, dilution 1:100) for 30 minutes at 4° C.; or without the anti. After this, three rounds of washing steps were performed using the staining buffer and cells were resuspended in 200 ul staining buffer and passed through a 40 μm cell strainer. Non-treated hCO that was not stained with CD90 served as a control for setting up the gate during acquisition of the cells. G575 was used for measuring PE signal, while R670 was used for measuring Cy5 signal. Flow cytometry was done on the BD Aria cell sorter in the Stanford Shared FACS Facility according to the Facility's calibration instructions. Data was processed using the FlowJo 10.7.1 software (BD).

Western blot for measuring protein level of CaV1.2. hCO derived from control and TS iPS cell lines were aliquoted to wells of 24-well ultra-low attachment plate (Corning; Cat. No. 3473). Each well contains 2-3 hCO cultured in 2 ml neural media. 1 μM ASO was added to the well. Media was half replaced at 3 days of ASO exposure. Samples were collected at 7 days of ASO exposure. Protein lysates for hCO's were prepared using RIPA buffer system (Santa Cruz, sc-24948). Protein lysates of t-hCO were prepared by briefly adding 50 μL of SDS Buffer (1.5% SDS, 25 mM Tris pH 7.5) for 2-3 organoids in a 1.5 mL tube, and then sonicate (Qsonica Q500 sonicator; pulse: 3 seconds on, 3 seconds off; amplitude: 20%). Protein concentrations were quantified using the Bicinchoninic Acid (BCA) assay (Pierce, ThermoFisher 23225). 20 μg of protein per sample per lane were loaded and run on a 4-12% Bis-Tris PAGE gel (Bolt 4-12% Bis-Tris Protein Gel, Invitrogen, NW04122BOX) and transferred onto a PVDF membrane (Immobulin-FL, EMD Millipore). Membranes were blocked with 5% BSA in TBST for 1 hour at room temperature (RT) and incubated with primary antibodies against GAPDH (mouse, 1:5000, GeneTex, GTX627408) overnight and CaV1.2 (rabbit, 1:1000, Alamone labs, ACC-003) for 48 hours for hCO samples and 96 hours for transplanted samples at 4° C. Membranes were washed 3 times with TBST and then incubated with near-infrared fluorophore-conjugated species-specific secondary antibodies: Goat Anti-Mouse IgG Polyclonal Antibody (IRDye 680RD, 1:10,000, LI-COR Biosciences, 926-68070) or Goat Anti-Rabbit IgG Polyclonal Antibody (IRDye 800CW, 1:10,000, LI-COR Biosciences, 926-32211) for 1 hour at RT. Following secondary antibody application, membranes were washed 3 times with TBST, once with TBS, and then imaged using a LI-COR Odyssey CLx imaging system (LI-COR).

Transplantation into athymic newborn rats. Animal procedures were performed by following animal care guidelines approved by Stanford University's Administrative Panel on Laboratory Animal Care (APLAC). Pregnant RNU euthymic (rnu/+) rats were purchased (Charles River Laboratories) or bred in house. Animals were maintained under a 12-hour light-dark cycle and provided food and water ad libitum. Three-to-seven-day-old athymic (FOXN1−/−) rat pups were identified by an immature whisker growth before culling. Pups (male and female) were anaesthetized with 2-3% isoflurane and mounted on a stereotaxic frame. A craniotomy, at about 2-3 mm in diameter, was performed above the S1, preserving the dura intact. Next, the dura mater was punctured using a 30-G needle (approximately 0.3 mm) close to the lateral side of the craniotomy. A hCO was next moved onto a thin 3×3-cm parafilm and excess media were removed. Using a Hamilton syringe connected to a 23 G, 450 needle, the hCO was gently pulled into the most distal tip of the needle. The syringe was next mounted on a syringe pump connected to the stereotaxic device. The sharp tip of the needle was next positioned above the 0.3-mm-wide pre-made puncture in the dura mater (z=0 mm), the syringe was reduced 1-2 mm (z=approximately −1.5 mm), and until a tight seal between the needle and the dura mater was formed. Next, the syringe was elevated to the center of the cortical surface at z=−0.5 mm, and the hCS was ejected at a speed of 1-2 μl per minute. After injection of hCO was completed, the needle was retracted at a rate of 0.2-0.5 mm per minute, the skin was closed, and the pups were immediately placed on a warmed heat pad until complete recovery.

MRI of transplanted rats. All animal procedures followed animal care guidelines approved by Stanford University's APLAC. Rats (more than 60 days post-transplantation) were anaesthetized with 5% isoflurane for induction and 1-3% isoflurane during imaging. For imaging, an actively shielded Bruker 7 Tesla horizontal bore scanner (Bruker Corp.) with International Electric Company (IECO) gradient drivers, a 120-mm inner diameter shielded gradient insert (600 mT/m, 1,000 T/m/s), AVANCE Ill electronics, eight-channel multi-coil radiofrequency and multinuclear capabilities, and the supporting Paravision 6.0.1 platform were used. Acquisitions were performed with an 86 mm inner diameter actively de-couplable volume radiofrequency coil with a four-channel cryo-cooled receive-only radiofrequency coil. Axial 2D Turbo-RARE (TR=2,500 ms, TE=33 ms, 2 averages) 16 slice acquisitions were performed with 0.6-0.8-mm slice thickness, with 256 Å˜256 samples. Signal was received with a 2-cm inner diameter quadrature transmit-receive volume radiofrequency coil (Rapid MR International, LLC). Successful transplantations were defined as transplantations that resulted in a continuous area of T2-weighted MRI signal in the transplanted hemisphere.

Cisterna magna injection of ASO to rats. Rats were anesthetized with 5% isoflurane for induction and 2-3% isoflurane during ASO injection through the cisterna magna (CM). Animals were set in a prone position with a small paper roll under the neck to tilt the head down. The neck was shaved and wiped off with ethanol. To target the CM, the foramen magnum was determined by touch and a 27G needle attached to syringe (BD, #305620) filled with 300 μg of ASO was percutaneously inserted into the CM perpendicularly to the neck. The needle was hold with the bevel face up and 30 μl of ASO was slowly injected into the CM. The procedure took <2 minutes per rat. Animals recovered from anesthesia within 10 minutes from the isoflurane induction.

Processing of ASO-injected rats. Rats were anaesthetized with isoflurane and brain tissue was removed and placed in cold (approximately 4° C.) oxygenated (95% O₂ and 5% CO₂) sucrose slicing solution containing: 234 mM sucrose, 11 mM glucose, 26 mM NaHCO₃, 2.5 mM KCl, 1.25 mM NaH₂PO₄, 10 mM MgSO₄ and 0.5 mM CaCl₂ (approximately 310 mOsm). Coronal rat brain slices (300-400 μm), containing t-hCO, were sectioned using a Leica VT1200 vibratome as previously described (3). t-hCO section were then moved to a continuously oxygenated slice chamber at RT, which contained aCSF: 10 mM glucose, 26 mM NaHCO₃, 2.5 mM KCl, 1.25 mM NaHPO4, 1 mM MgSO4, 2 mM CaCl₂ and 126 mM NaCl (298 mOsm).

Calcium imaging on t-hCO from rats receiving ASO injection. Post-sectioning and dissecting of the t-hCO from the rats, the slices were incubated with Calbryte™ 520 AM (AAT Bioquest, Cat. No. 20650) in 1:1 of NPC media and PBS for 45 minutes to 1 hour at 37° C. The slices were then transfer to a 24-well imaging plate containing 500 μl warm low-potassium Tyrode's solution (5 mM KCl, 129 mM NaCl, 2 mM CaCl₂, 1 mM MgCl₂, 30 mM glucose, 25 mM HEPES, pH 7.4) and imaged with a confocal microscope (Leica Stellaris) for 30 s at 37° C., after which media were replaced by high-potassium Tyrode's solution (high-KCl; 67 mM KCl: 67 mM NaCl, 2 mM CaCl₂, 1 mM MgCl₂, 30 mM glucose and 25 mM HEPES, pH 7.4) and imaging was restored. The imaging continues for a total period of 25 minutes. Mean gray values were collected from ROIs delineating Calbryte⁺ somas (visualized by the standard deviation projection of the entire timeseries) with Fiji (ImageJ, version 2.1.0, NIH). Mean gray values were transformed to relative changes in fluorescence: dF/F(t)=(F(t) FO)/FO, where FO represents average grey values of the time series of each ROI). Residual calcium was calculated as (C-A)/(B-A) where B is the peak value following depolarization (max peak value determined by custom-written MATLAB routines (version R2019b, 9.4.0, MathWorks), A is the baseline value (B-50th frame) and C is the decay value (B+150th frame).

Golgi staining. Golgi staining was conducted using the FD Rapid GolgiStain Kit (FD Neurotechnologies, Cat. No. PK401) according to manufacturer's instructions. In brief, freshly dissected t-hCOs were incubated with solution A/B mixture in the dark, then transferred into solution C. After 72 hours, the tissue was embedded in agarose, vibratome chamber filled with solution C, and sectioned at 100 μm using a Leica VT1200S vibratome. Sections were mounted on gelatin coated slides, stained in solution D/E, washed, dehydrated, cleared, and cover-slipped. Images were acquired on a SP8 confocal microscope with bright field. Cells were counted as neurons based on their morphology. Dendrites were manually traced using neuTube. Both the tracing and the analysis were performed blinded.

Statistics. Data are presented as mean±S.E.M., unless otherwise indicated. Distribution of the raw data was tested for normality of distribution; statistical analyses were performed using the Student's t-test, one-way analysis of variance (ANOVA) with multiple comparisons. Statistical analyses were performed in Prism (GraphPad). Data shown from representative experiments were repeated with similar results in at least three independent biological replicates, unless otherwise noted. Sample sizes were estimated empirically.

### REFERENCES

1 Splawski, I. et al. Severe arrhythmia disorder caused by cardiac L-type calcium channel mutations. Proc Natl Acad Sci USA 102, 8089-8096; discussion 8086-8088, doi:10.1073/pnas.0502506102 (2005).
2 Paşca, S. P. et al. Using iPSC-derived neurons to uncover cellular phenotypes associated with Timothy syndrome. Nature Medicine 17, 1657-1662, doi:10.1038/nm.2576 (2011).
3 Birey, F. et al. Assembly of functionally integrated human forebrain spheroids. Nature 545, 54-59, doi:10.1038/nature22330 (2017).
4 Birey, F. et al. Dissecting the molecular basis of human interneuron migration in forebrain assembloids from Timothy syndrome. Cell Stem Cell 29, 248-264.e247, doi:10.1016/j.stem.2021.11.011 (2022).
5 Panagiotakos, G. et al. Aberrant calcium channel splicing drives defects in cortical differentiation in Timothy syndrome. eLife 8, e51037, doi:10.7554/eLife.51037 (2019).
6 Revah, O. et al. Maturation and circuit integration of transplanted human cortical organoids. Nature (2022).
7 Splawski, I. et al. Ca(V) 1.2 calcium channel dysfunction causes a multisystem disorder including arrhythmia and autism. Cell 119, 19-31, doi:10.1016/j.cell.2004.09.011 (2004).
8 Napolitano, C., Timothy, K. W., Raffaella, B. & P. G., S. in GeneReviews (2006).
9 Dufendach, K. A. et al. Clinical Outcomes and Modes of Death in Timothy Syndrome: A Multicenter International Study of a Rare Disorder. JACC Clin Electrophysiol 4, 459-466, doi:10.1016/j.jacep.2017.08.007 (2018).
10 Levy, R. J. et al. A cross-sectional study of the neuropsychiatric phenotype of CACNA1C-related disorder. medRxiv, doi:10.1101/2022.06.22.22276784 (2022).
11 Yazawa, M. et al. Using induced pluripotent stem cells to investigate cardiac phenotypes in Timothy syndrome. Nature 471, 230-234, doi:10.1038/nature09855 (2011).
12 Welling, A. et al. Alternatively spliced IS6 segments of the alpha 1C gene determine the tissue-specific dihydropyridine sensitivity of cardiac and vascular smooth muscle L-type Ca2+ channels. Circulation Research 81, 526-532, doi:doi:10.1161/01.RES.81.4.526 (1997).
13 Zühlke, R. D., Bouron, A., Soldatov, N. M. & Reuter, H. Ca2+ channel sensitivity towards the blocker isradipine is affected by alternative splicing of the human α1C subunit gene. FEBS Letters 427, 220-224, doi:https://doi.org/10.1016/S0014-5793(98) 00425-6 (1998).
14 Rinaldi, C. & Wood, M. J. A. Antisense oligonucleotides: the next frontier for treatment of neurological disorders. Nature Reviews Neurology 14, 9-21, doi:10.1038/nrneurol.2017.148 (2018).
15 Crooke, S. T., Baker, B. F., Crooke, R. M. & Liang, X. h. Antisense technology: an overview and prospectus. Nature Reviews Drug Discovery 20, 427-453, doi:10.1038/s41573-021-00162-z (2021).
16 Bader, P. L. et al. Mouse model of Timothy syndrome recapitulates triad of autistic traits. Proceedings of the National Academy of Sciences 108, 15432-15437, doi:doi:10.1073/pnas.1112667108 (2011).
17 Paşca, A. M. et al. Functional cortical neurons and astrocytes from human pluripotent stem cells in 3D culture. Nat Methods 12, 671-678, doi:10.1038/nmeth.3415 (2015).
18 Sloan, S. A., Andersen, J., Paşca, A. M., Birey, F. & Paşca, S. P. Generation and assembly of human brain region-specific three-dimensional cultures. Nature Protocols 13, 2062-2085, doi:10.1038/s41596-018-0032-7 (2018).
19 Barreto-Chang, O. L. & Dolmetsch, R. E. Calcium imaging of cortical neurons using Fura-2 AM. J Vis Exp, doi:10.3791/1067 (2009).
20 Khan, T. A. et al. Neuronal defects in a human cellular model of 22q11.2 deletion syndrome. Nat Med 26, 1888-1898, doi:10.1038/s41591-020-1043-9 (2020)
21 Revah O, Gore F, Kelley K W, Andersen J, Sakai N, Chen X, Li M Y, Birey F, Yang X, Saw N L, Baker S W, Amin N D, Kulkarni S, Mudipalli R, Cui B, Nishino S, Grant G A, Knowles J K, Shamloo M, Huguenard J R, Deisseroth K, Paşca S P. Maturation and circuit integration of transplanted human cortical organoids. Nature. 2022 October; 610(7931):319-326. doi: 10.1038/s41586-022-05277-w. Epub 2022 Oct. 12. PMID: 36224417; PMCID: PMC9556304.

Notwithstanding the appended clauses, the disclosure set forth herein is also described by the following clauses:
1. A method for treating an individual having Timothy syndrome, the method comprising: administering an effective dose of an agent to the individual, wherein the agent modulates the splicing of an 8 A or an 8 exon of CACNA1C.
2. The method of clause 1, wherein the agent is a nucleic acid.
3. The method of clause 2, wherein the nucleic acid is an antisense oligonucleotide (ASO).
4. The method of clause 3, wherein the ASO comprises a 2′-O-methoxyethylribose modification.
5. The method of clause 2, wherein the nucleic acid is a double-stranded silencing RNA (siRNA).
6. The method of any of the preceding clauses, wherein the Timothy syndrome is type-1 Timothy syndrome.
7. The method of clause 6, wherein the individual has a G406R amino acid substitution in a Caᵥ1.2α subunit encoded by CACNA1C.
8. The method of clauses 6 or 7, wherein the agent inhibits the splicing of the 8 A exon of CACNA1C.
9. The method of any of clauses 6-8, wherein the agent comprises a sequence selected from the group of AAATAGATCCAGGGCCAGTC (SEQ ID NO 14; ASO14), CAGTCCCTTCCTACGGCATC (SEQ ID NO 15; ASO17), and CCTTCCTACGGCATCATTGA (SEQ ID NO 16; ASO18).
10. The method of any of clauses 6-9, wherein the treatment results in a reduction of residual Ca2+ levels in a human neuron following neuronal depolarization.
11. The method of any of clauses 6-10, wherein the treatment results in decreased expression of the 8 A exon of CACNA1C.
12. The method of any of clauses 1-5, wherein the Timothy syndrome is type-2 Timothy syndrome.
13. The method of clause 12, wherein the agent inhibits the splicing of the 8 exon of CACNA1C.
14. The methods of clauses 12 or 13, wherein the treatment results in decreased expression of the 8 exon.
15. The method of any of the preceding clauses, wherein the treatment does not reduce total amount of a Caᵥ1.2 protein.
16. The method of any of the preceding clauses, further comprising genotyping the individual to determine if the individual has a mutation associated with Timothy syndrome prior to the administration.
17. The method of any of the preceding clauses, wherein two or more agents are administered to the individual.
18. The method of any of the preceding clauses, wherein the agent is administered locally.
19. The method of any of the preceding clauses, wherein the agent is administered systemically.
20. A composition, the composition comprising:
   the agent of clause 1, and
   a pharmaceutically expectable excipient.
21. The composition of clause 20, further comprising a second agent.
22. The composition of clause 21, wherein the second agent is a nucleic acid.
23. The composition of clause 22, wherein the nucleic acid is an antisense oligonucleotide (ASO).
24. The composition of any of clauses 20-23, wherein the ASO comprises a 2′-O-methoxyethylribose modification.
25. The composition of clause 22, wherein the nucleic acid is a double-stranded silencing RNA (siRNA).
26. The composition of any of clauses 20-25, wherein the agent modulates the splicing of the 8 A exon of CACNA1C.
27. The composition of any of clauses 20-26, wherein the agent comprises a sequence selected from the group of AAATAGATCCAGGGCCAGTC (SEQ ID NO 14; ASO14), CAGTCCCTTCCTACGGCATC (SEQ ID NO 15; ASO17), and CCTTCCTACGGCATCATTGA (SEQ ID NO 16; ASO18).
28. The composition of any of clauses 20-26, wherein the individual has a G406R amino acid substitution in a Caᵥ1.2α subunit encoded by CACNA1C.
29. The composition of any of clauses 20-28 wherein the treatment results in a reduction of residual Ca2+ levels in a neuron following neuronal depolarization.
30. The composition of any of clauses 20-29, wherein the treatment results in decreased expression of the 8 A exon of CACNA1C.
31. The composition of any of clauses 20-24, wherein the agent inhibits the splicing of the 8 exon of CACNA1C.
32. The composition of clause 31, wherein the treatment results in decreased expression of the 8 exon.
33. The composition of any of clauses 20-32, wherein the treatment does not reduce total amount of a Caᵥ1.2 protein.
34. A kit, the kit comprising:
   the composition of any of the preceding clauses.
35. A method of screening a candidate agent for activity in treating a condition associated with a defect in gene splicing, the method comprising:
   (i) administering the candidate agent to human tissue derived from an individual having the condition associated with the defect in gene splicing,
   (ii) measuring the expression of an exon that is associated with the condition associated with the defect in gene splicing in the human tissue, and
   (iii) comparing the expression of the exon that is associated with the condition associated with the defect in gene splicing in the human tissue in the absence of the administration of the candidate agent.
36. The method of clause 35, wherein the condition is a neuropsychiatric condition.
37. The method of clause 36 wherein the neuropsychiatric condition is selected from the group consisting of Parkinson's disease, Alzheimer's disease, Amyotrophic lateral sclerosis (ALS), frontotemporal dementia (FTD), Familial dysautonomia (FD), and Timothy syndrome.
38. The method of any of clauses 35-37, wherein the individual having the neuropsychiatric condition associated with the defect in gene splicing has a genetic mutation that results in the defect in gene splicing.
39. The method of any of clauses 35-38, wherein the candidate agent is selected from the group consisting of a nucleic acid, a small molecule, a protein, or a compound.
40. The method of any of clauses 35-39, wherein the human tissue is human neural tissue.
41. The method of any of clauses 35-39, wherein the human tissue is human cardiac tissue.
42. The method of any of clauses 35-39, wherein the human tissue is human gastrointestinal tissue.
43. The method of any of clauses 35-39, wherein the human tissue is human lung tissue.
44. The method of any of clauses 35-39, wherein the human tissue is human liver tissue.
45. The method of any of clauses 35-39, wherein the human tissue is human kidney tissue.
46. The method of any of clauses 35-39, wherein the human tissue is human vascular tissue.
47. The method of any of clauses 35-39, wherein the human tissue is human retina tissue.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

That which is claimed is:

1. A method for treating an individual having Timothy syndrome, the method comprising:
administering an effective dose of an agent to the individual, wherein the agent modulates the splicing of an 8 A or an 8 exon of CACNA1C.

2. The method of claim 1, wherein the agent is a nucleic acid.

3. The method of claim 2, wherein the nucleic acid is an antisense oligonucleotide (ASO).

4. The method of claim 3, wherein the ASO comprises a 2′-O-methoxyethylribose modification.

5. The method of claim 2, wherein the nucleic acid is a double-stranded silencing RNA (siRNA).

6. The method of claim 1, wherein the Timothy syndrome is type-1 Timothy syndrome.

7. The method of claim 6, wherein the individual has a G406R amino acid substitution in a Caᵥ1.2α subunit encoded by CACNA1C.

8. The method of claim 1, wherein the agent inhibits the splicing of the 8 A exon of CACNA1C.

9. The method of claim 1, wherein the agent comprises a sequence selected from the group of AAATAGATCCAGGGCCAGTC (SEQ ID NO 14; ASO14), CAGTCCCTTCCTACGGCATC (SEQ ID NO 15; ASO17), and CCTTCCTACGGCATCATTGA (SEQ ID NO 16; ASO18).

10. The method of claim 1, wherein the treatment results in a reduction of residual Ca²⁺ levels in a human neuron following neuronal depolarization.

11. The method of claim 1, wherein the treatment results in decreased expression of the 8 A exon of CACNA1C.

12. The method of claim 1, wherein the Timothy syndrome is type-2 Timothy syndrome.

13. The method of claim 1, wherein the agent inhibits the splicing of the 8 exon of CACNA1C.

14. The method of claim 1, wherein the treatment results in decreased expression of the 8 exon.

15. The method of claim 1, wherein the treatment does not reduce total amount of a Cav1.2 protein.

16. The method of claim 1, further comprising genotyping the individual to determine if the individual has a mutation associated with Timothy syndrome prior to the administration.

17. The method of claim 1, wherein two or more agents are administered to the individual.

18. The method of claim 1, wherein the agent is administered locally.

19. The method of claim 1, wherein the agent is administered systemically.

20. A composition comprising:
the agent of claim 1, and
a pharmaceutically expectable excipient.


SEQUENCE LISTING (36 sequences)

SEQ ID NO: 1 - RNA, 20 nt, synthetic construct
  source 1..20: mol_type = other RNA
tccttggtcctgcttacctg

SEQ ID NO: 2 - RNA, 20 nt, synthetic construct
  source 1..20: mol_type = other RNA
ggtcctgcttacctgctaag

SEQ ID NO: 3 - RNA, 20 nt, synthetic construct
  source 1..20: mol_type = other RNA
tgcttacctgctaagcacac

SEQ ID NO: 4 - RNA, 20 nt, synthetic construct
  source 1..20: mol_type = other RNA
acctgctaagcacaccgaga

SEQ ID NO: 5 - RNA, 20 nt, synthetic construct
  source 1..20: mol_type = other RNA
ctaagcacaccgagaaccaa

SEQ ID NO: 6 - RNA, 20 nt, synthetic construct
  source 1..20: mol_type = other RNA
cacaccgagaaccaagttaa

SEQ ID NO: 7 - RNA, 20 nt, synthetic construct
  source 1..20: mol_type = other RNA
cgagaaccaagttaagtaca

SEQ ID NO: 8 - RNA, 20 nt, synthetic construct
  source 1..20: mol_type = other RNA
accaagttaagtacaaaaaa

SEQ ID NO: 9 - RNA, 20 nt, synthetic construct
  source 1..20: mol_type = other RNA
tgaccctatgatgattagtg

SEQ ID NO: 10 - RNA, 20 nt, synthetic construct
  source 1..20: mol_type = other RNA
ctatgatgattagtgtaaca

SEQ ID NO: 11 - RNA, 20 nt, synthetic construct
  source 1..20: mol_type = other RNA
atgattagtgtaacaaaata

SEQ ID NO: 12 - RNA, 20 nt, synthetic construct
  source 1..20: mol_type = other RNA
tagtgtaacaaaatagatcc

SEQ ID NO: 13 - RNA, 20 nt, synthetic construct
  source 1..20: mol_type = other RNA
taacaaaatagatccagggc

SEQ ID NO: 14 - RNA, 20 nt, synthetic construct
  source 1..20: mol_type = other RNA
aaatagatccagggccagtc

SEQ ID NO: 15 - RNA, 20 nt, synthetic construct
  source 1..20: mol_type = other RNA
cagtcccttcctacggcatc

SEQ ID NO: 16 - RNA, 20 nt, synthetic construct
  source 1..20: mol_type = other RNA
ccttcctacggcatcattga

SEQ ID NO: 17 - RNA, 20 nt, synthetic construct
  source 1..20: mol_type = other RNA
ctacggcatcattgacctgg

SEQ ID NO: 18 - RNA, 20 nt, synthetic construct
  source 1..20: mol_type = other RNA
gcatcattgacctggaagaa

SEQ ID NO: 19 - RNA, 20 nt, synthetic construct
  source 1..20: mol_type = other RNA
attgacctggaagaacggaa

SEQ ID NO: 20 - RNA, 20 nt, synthetic construct
  source 1..20: mol_type = other RNA
cctggaagaacggaatgata

SEQ ID NO: 21 - RNA, 29 nt, Homo sapiens
  source 1..29: mol_type = mRNA
ccaggtcaatgatgccgcttagcgggtaa

SEQ ID NO: 22 - RNA, 29 nt, Homo sapiens
  source 1..29: mol_type = mRNA
ccaggtcaatgatgccgcttagcaggtaa

SEQ ID NO: 23 - DNA, 140 bp, Homo sapiens
  source 1..140: mol_type = genomic DNA
tatcattccgttcttccaggtcaatgatgccgtaggaagggactggccctggatctattttgttacactaatcatcatagggtcattttttgtacttaacttggttctcggtgtgcttagcaggtaagcaggaccaagga

SEQ ID NO: 24 - DNA, 73 bp, synthetic construct
  source 1..73: mol_type = other DNA
ggatctattttgttacactaatcatcatagggtcatttttgtscttaacttggttctcggtgtgcttagcagg

SEQ ID NO: 25 - DNA, 74 bp, synthetic construct
  source 1..74: mol_type = other DNA
ggatctattttgttacactaatcatcatagggtcattttttgtacttaacttggttctcggtgtgcttagcggg

SEQ ID NO: 26 - DNA, 74 bp, synthetic construct; identical to SEQ ID NO: 25
  source 1..74: mol_type = other DNA

SEQ ID NO: 27 - DNA, 74 bp, synthetic construct
  source 1..74: mol_type = other DNA
ggatctattttgttacactaatcatcatagggtcattttttgtacttaacttggttctcggtgtgcttagcagg

SEQ ID NO: 28 - DNA, 74 bp, synthetic construct; identical to SEQ ID NO: 27
  source 1..74: mol_type = other DNA

SEQ ID NO: 29 - DNA, 74 bp, synthetic construct; identical to SEQ ID NO: 27
  source 1..74: mol_type = other DNA

SEQ ID NO: 30 - DNA, 74 bp, synthetic construct; identical to SEQ ID NO: 27
  source 1..74: mol_type = other DNA

SEQ ID NO: 31 - DNA, 104 bp, Rattus norvegicus
  source 1..104: mol_type = genomic DNA
gtcaatgatgccgtaggaagggactggccctggatctattttgtaacactaatcatcatagggtcattttttgtacttaacttggttctcggtgttcttagcgg

SEQ ID NO: 32 - DNA, 104 bp, Homo sapiens
  source 1..104: mol_type = genomic DNA
gtcaatgatgccgtaggaagggactggccctggatctattttgttacactaatcatcatagggtcattttttgtacttaacttggttctcggtgtgcttagcgg

SEQ ID NO: 33 - DNA, 22 bp, synthetic construct
  source 1..22: mol_type = other DNA
cttctttcctaactttccttcg

SEQ ID NO: 34 - DNA, 21 bp, synthetic construct
  source 1..21: mol_type = other DNA
ctgcgttgtggagaggacata

SEQ ID NO: 35 - DNA, 22 bp, synthetic construct
  source 1..22: mol_type = other DNA
gtgcctcactaactatcattcc

SEQ ID NO: 36 - DNA, 23 bp, synthetic construct
  source 1..23: mol_type = other DNA
aaatcaagacctttttccttggt